(19) United States
(12) Patent Application Publication
(10) Pub. No.: US 2005/0142077 A1
(43) Pub. Date: Jun. 30, 2005

(54) Usa of an antimicrobial glass ceramic for dental care and oral hygiene

(76) Inventors: Jose Zimmer, Ingelheim (DE); Jorg Hinrich Fechner, Mainz (DE)

Correspondence Address: OHLANDT, GREELEY, RUGGIERO & PERLE, LLP, ONE LANDMARK SQUARE, 10TH FLOOR, STAMFORD, CT, 06901, US

(21) Appl. No.: 10/498,167

(22) PCT Filed: Dec. 11, 2002
(86) PCT No.: PCT/EP02/14042

(30) Foreign Application Priority Data
Dec. 12, 2001 (DE) 101 61 074.2
Sep. 7, 2002 (DE) 102 41 496.3

(51) Int. Cl. A61K 7/16; C03C 3/19; A61C 5/00

(52) U.S. Cl. 424/57; 433/217.1; 501/47

(57) ABSTRACT

A method of using glass ceramic in oral hygiene and/or dental care is provided. The method includes applying a composition to the mouth of a user. The composition includes SiO₂ 30 to 70 weight-percent; Na₂O 0 to 40 weight-percent; K₂O 0 to 40 weight-percent; CaO 5 to 40 weight-percent; MgO 0 to 40 weight-percent; Al₂O₃ 0 to 5 weight-percent; P₂O₅ 0 to 20 weight-percent; B₂O₃ 0 to 5 weight-percent; ZnO 0 to 10 weight-percent; and 0 to 30 weight-percent Xfy, wherein X is selected from the group consisting of Na, K, Mg, and Ca, y is equal to 1 or 2, and the sum of Na₂O+K₂O+CaO is greater than 10 weight-percent and less than 70 weight-percent.

The object of the present invention is the usage of an antimicrobial, anti-inflammatory, remineralizing, tartar-reducing glass ceramic and/or an antimicrobial glass ceramic powder in dental care. A possible starting glass for a glass ceramic and/or a glass ceramic powder of this type includes 30-75 weight-percent SiO₂, 0-40 weight-percent Na₂O, 0-40 weight-percent CaO, and 0-20 weight-percent P₂O₅, with the sum Na₂O+K₂O+CaO<70 weight-percent and >10 weight-percent.

Glasses having a bioactive and sometimes also antimicrobial effect are described as bioglass in L. L. Hensch, J. Wilson, An Introduction to Bioceramics, World Scientific Publ., 1993. Bioglass of this type is distinguished by the formation of hydroxylapatite layers in aqueous media. Heavy-metal-free alkali-alkaline earth silicate glasses having antimicrobial properties are described in the applications DE-A-199 32 238 and DE-A-199 32 239.

A glass powder which includes 40-60 weight-percent SiO₂, 5-30 weight-percent Na₂O, 10-35 weight-percent CaO, and 0-12 weight-percent P₂O₅ is known from U.S. Pat. No. 5,676,720, a glass ceramic manufactured from a glass of this type of composition also being known. However, no information about the crystal phase is given in U.S. Pat. No. 5,676,720.

U.S. Pat. No. 5,981,412 describes a bioactive bioceramic for medical applications having the crystalline phase Na₂O.2CaO.3SiO₂. The crystallite size is 13 μm. The ceramization is performed using tempering steps for nucleation and crystallization. The emphasis is on the mechanical properties such as K_{1c}. The crystal phase component is between 34 and 60 volume-percent. U.S. Pat. No. 5,981,412 only describes a crystalline phase which is a high-temperature phase and which only arises under the special conditions specified in this publication. An application in the field of dental care is not described.

The use of bioactive glasses for toothpaste and gels is described, for example, in WO 97/27148. Inorganic non-metallic materials which contain calcium and phosphorus and lead to remineralization of the teeth through appropriate ion donations are known from U.S. Pat. Nos. 5,427,768 and 5,268,167.

The use of the glasses cited above in the fields of dental care and/or oral hygiene has the disadvantage that ions are only released in an uncontrolled way and the abrasive effect of these glasses (their use as a polishing agent) is not adjustable.

Therefore, the object arises of specifying materials for dental care and/or oral hygiene which do not have the disadvantages of the related art, in particular, the materials are to have an abrasive effect which may be adjusted in a defined way. In addition, the materials are to have an antimicrobial effect. Furthermore, these materials are to cause accelerated remineralization of the teeth and reduce the formation of tartar and strengthen the gums through the anti-inflammatory effect.

The inventors have now found out that the disadvantages of the related art may be overcome if the glass ceramics or glass ceramic powders described in the present invention are used in the fields of dental care and/or oral hygiene. In the present invention, glass ceramic powders also include glass ceramic fibers, balls (diameter <100 μm, preferably less than 20 μm) and glass ceramic beads (diameter<100 μm, preferably less than 20 μm).

The glass ceramic according to the present invention may be used in the fields of dental care and/or oral hygiene as a polishing agent for teeth, an agent against tartar, a desensitization agent for sensitive teeth, to prevent the formation of acids in the oral region, and therefore to reduce caries formation, and, through its antimicrobial effect, to reduce the mouth odor and particularly for remineralization of the teeth and/or for the construction of a mineral layer on the tooth surface.

A glass ceramic and/or a powder made of a glass ceramic of this type, which, besides the antimicrobial properties, also has anti-inflammatory, remineralizing, tartar-reducing, and desensitizing properties, is especially suitable.

Glass ceramics, particularly glass ceramic powders, are especially preferred in which the starting glass has the following composition in weight-present in relation to the oxide:

| | | |
|---|---|---|
| SiO₂ | 30-70 | weight-percent |
| Na₂O | 0-40 | weight-percent |
| K₂O | 0-40 | weight-percent |
| CaO | 5-40 | weight-percent |
| MgO | 0-40 | weight-percent |
| Al₂O₃ | 0-5 | weight-percent |
| P₂O₅ | 0-20 | weight-percent |
| B₂O₃ | 0-5 | weight-percent |
| ZnO | 0-10 | weight-percent |

and

0 to 30 weight-percent XFy, X able to be Na, K, Mg, or Ca and y=1 or y=2 and the sum of Na₂O+K₂O+CaO particularly being 5-70 weight-percent.

In an alternative embodiment, the starting glass has

| | | |
|---|---|---|
| SiO₂ | 30-60 | weight-percent |
| K₂O | 0-30 | weight-percent |
| Na₂O | 5-30 | weight-percent |
| CaO | 5-30 | weight-percent |
| P₂O₅ | 2-10 | weight-percent |
| ZnO | 0-10 | weight-percent |

and

0 to 30 weight-percent XFy, X able to be Na, K, Mg, or Ca and y=1 or y=2, in weight-percent in relation to the oxide.

In a further embodiment, the starting glass has the following composition:

| | | |
|---|---|---|
| SiO₂ | 30-60 | weight-percent |
| K₂O | 5-30 | weight-percent |
| Na₂O | 0-30 | weight-percent |
| CaO | 5-30 | weight-percent |
| P₂O₅ | 2-10 | weight-percent |
| ZnO | 0-10 | weight-percent |

and

0 to 30 weight-percent XFy, X able to be Na, K, Mg, or Ca and y=1 or y=2, in weight-percent in relation to the oxide.

The crystalline primary phase of the glass ceramic obtained from the starting glasses cited above is especially preferably made of alkali-alkaline earth silicates and/or alkaline earth silicates and/or alkali silicates.

The glass ceramic and/or the glass ceramic powder according to the present invention are distinguished in that they have a biocidal effect, or at least a biostatic effect, on bacteria and fungi. The glass ceramic according to the present invention is tolerated by the mucosa and toxicologically harmless when in contact with humans.

If the glass ceramic according to the present invention is used in the fields of dental care and/or oral hygiene, the maximum concentration of heavy metals is, for example, <20 ppm for Pb, <5 ppm for Cd, <5 ppm for As, <10 ppm for Sb, <1 ppm for Hg, and <10 ppm for Ni.

In this case, exceptions may be colored glasses which are produced through doping using coloring ions or colloids, for example.

All of the statements in the application are in relation to weight-percent based on the oxide.

The unceramized starting glass which is used for manufacturing the glass ceramic according to the present invention contains between 30-70 weight-percent SiO₂ as the network former. At lower concentrations, the spontaneous tendency to crystallize increases strongly and the chemical resistance falls strongly. At higher SiO₂ values, the crystallization stability may also drop and the processing temperature is significantly increased, so that the hot shaping properties worsen. SiO₂ is also a component of the crystalline phases arising during ceramization and must be contained in the glass in appropriately high concentrations if high crystalline phase components are to be achieved to the ceramization.

Na₂O is used as the flux during the melting of the glass. At concentrations lower than 5 percent, the melting behavior is negatively influenced in the absence of further alkaline oxides. Sodium is a component of the phases formed during ceramization and must be contained in the glass in appropriately high concentrations if high crystalline phase components are to be achieved through the ceramization.

K₂O acts as a flux during the melting of the glass. In addition, potassium is released in aqueous systems. If there are high potassium concentrations in the glass, potassium-containing phases such as potassium silicates are also separated. The sum of the alkalis used to be greater than 5 weight-percent to ensure the meltability. The K₂O content may be in the range 0-40 weight-percent, preferably 0-25 weight-percent, especially preferably 0-10 weight-percent.

The chemical resistance of the glass and therefore the ion donation in aqueous media is set via the P₂O₅ content. The P₂O₅ content is between 0 and 15 weight-percent. At higher P₂O₅ values, the hydrolytic resistance of the glass ceramic is too low.

In order to improve the meltability, the glass may have up to 5 weight-percent B₂O₃.

The quantity of Al₂O₃ is to be less than 5 weight-percent, in order to achieve a chemical resistance which is not too high. Al₂O₃ is used to set the chemical resistance of the glass.

To amplify the antimicrobial, particularly the antibacterial properties of the glass ceramic, antimicrobially acting ions such as Ag, Au, I, Ce, Cu, Zn, and Sn may be included in concentrations of less than 5 weight-percent.

Furthermore, ions such as Ag, Cu, Au, and Li may be included as additives to set the high temperature conductivity of the melts and therefore for improved meltability using high frequency melting methods. The concentration of these ions is to be less than 5 weight-percent.

Coloring ions such as Fe, Cr, Co, V, Cu, Mn, and Ag may be included individually or combined in a total concentration less than 1 weight-percent.

For tooth hardening, the base glass used for the glass ceramic may contain 0-30 weight-percent XFy, X able to be Na, K, Mg, or Ca and y=1 or y=2.

Typically, the glass ceramic according to the present invention is used in powder form. The ceramization of the starting glass may either be performed using a glass block and/or glass ribbon or even using glass powder. After the ceramization, the glass ceramic blocks or ribbons must be milled into powder. If the powder has been ceramicized, it must also be milled again if necessary, in order to remove agglomerates which have arisen during the ceramization step. The milling may be performed either dry or even in aqueous and non-aqueous milling media.

The decisive advantage of the ceramization in powder form is a very small crystallite size while still having high total phase components. In addition, the crystallites grow from the surface on surface defects which are generated during milling. The tribological activation of the surface elevates the reactivity of the powder.

A very large number of surface nuclei are generated through milling, so that a very large number of crystals begin to grow simultaneously and therefore an extremely small crystallite size may be achieved while nonetheless having high crystalline phase components. A separate additional tempering treatment for nucleation, as described in U.S. Pat. No. 5,981,412, for example, is therefore not necessary.

The particle sizes are typically smaller than 500 μm. Particle sizes <100 μm and/or <20 μm have been shown to be expedient. Particle sizes <10 μm and smaller than 5 μm and smaller than 2 μm are especially suitable. Particle sizes <1 μm have been shown to be very especially suitable.

Mixtures of different glass powders from the composition range having different compositions and grain sizes are possible in order to combine specific effects.

If a block or a ribbon of a starting glass is ceramicized, the crystallite sizes are in the range greater than 10 μm if crystalline phase components of greater than 30 volume-percent are desired. The crystallization runs very rapidly. The ceramization temperatures are from 50° C. to 400° C. above Tg. The ceramization may also be performed in multistage thermal processes in this case. The crystallization is primarily surface-controlled. Needle-shaped crystallites grow from the surface into the inside of the glass. A few crystallites also begin to grow inside of the glass. They are predominantly spherulitic. Needle-shaped crystals primarily arise during the ceramization of the powder because of the large surfaces.

The ceramization of the starting glass is surface-controlled. If the ribbons and/or blocks of the starting glass are milled into powders before the ceramization, the crystallization temperatures shift to significantly lower values. The crystals begin to grow from the surfaces of the powder particles into the inside. The ceramization may be guided in such a way that the particles only have an outer crystalline layer and remain amorphous inside. The selection of the particle size determines the average crystallite size. Through partial crystallization of the particles on the surface, special effects may be achieved during the reaction and ion donation of the partially-ceramicized powder. For example, if a phase is generated on the surface which is more reactive than the glass, short-term and long-term behaviors of the ion donation may be combined.

The crystal phase components in the glass after the ceramization are greater than 5 weight-percent. Depending oh the composition of the starting glass, up to nearly 100 weight-percent crystalline phase components may be reached. Preferred ranges are phase components greater than 10 weight-percent and greater than 30 weight-percent. The range greater than 50 weight-percent is even more preferable.

Depending on the ceramization temperature, the ceramicized powder is milled again in order to break up agglomerations which have arisen during the ceramization once again.

Crystal primary phases are alkali-alkaline earth silicates and/or alkaline earth silicates, particularly Na—Ca silicates and Ca silicates, these phase components able to be influenced through the ceramization.

Further crystal secondary phases, which may contain silver and/or phosphorus and/or silicon, such as AgPO₃, SiP₂O₇, or SiO₂, may also arise depending on the special composition of the starting glass.

Glass ceramics containing phosphorus from this composition range may be bioactive in aqueous media, i.e., they form a hydroxylapatite layer on their surface and also on external surfaces in aqueous systems. Therefore, such powders are especially suitable for being used as biomaterials or in applications in which remineralization processes play an important role, i.e., in the field of dental care, for example.

The chemical reactivity and/or the ion donation are influenced by the phases and phase components. Chemical reactivity and ion donation may therefore be controlled so that via these the primary compatibility, the pH value, and antimicrobial and anti-inflammatory effects may be adjusted.

The crystalline phases show a significantly different chemical resistance than the glass phase. The chemical phase may be both increased and decreased. Besides the chemical properties, the mechanical, abrasive, and optical properties are also modified in accordance with the crystal primary phase properties.

At relatively low ceramization temperatures <700° C., one to two Na—Ca silicates are first formed in ribbons. These are preferably (Na₂CaSi₃O₈/Na₂CaSiO₄)/Na₂Ca₂(SiO₃)₃. At temperatures greater than 700° C., recrystallization occurs.

The resulting crystalline phases sometimes show a significantly higher solubility in water than the glass phase. By setting the phase components in a targeted way, the ion donation of the powder and the pH value in aqueous solution, and therefore the also the biological effect, may be influenced.

If the crystalline phase is dissolved in water or aqueous solution, honeycombed and/or porous surface structures remain, which particularly influence optical properties such as transmission, reflection, and light scattering of the powder in formulations. In partial solutions in aqueous systems, formation of nanoparticles is also observed.

The glass ceramic powders according to the present invention may be used in multiple formulations for dental care and/or oral hygiene. Toothpastes are cited here only as examples. By dissolving the crystalline phases, nanoparticles arise. These nanoparticles may seal tubulin channels in the tooth, among other things, and therefore have a desensitizing effect on sensitive teeth. The formulations for dental care and/or oral hygiene, in which the glass ceramic according to the present invention is used, may be non-aqueous or even contain water. It is especially preferable if the particle size of the glass powder is smaller than 10 μm. If the glass ceramic and/or glass ceramic powder according to the present invention is used in formulations for oral hygiene and oral care, because of the ceramization, in contrast to the use of pure glass powder, the powders and therefore the formulations may have their reactivity set in a defined way, and may particularly be more reactive than the corresponding glasses. If a glass ceramic powder is used instead of a glass powder, the formation of the hydroxylapatite coating and the ion donation are significantly accelerated. Significant advantages over the glass powders used in the field of dental care result therefrom, since only a relatively short action time is necessary. In formulations for dental care, the glass ceramic powder according to the present invention may effectively prevent bleeding gums and periodontitis as well as suppress mouth odor and whiten teeth. Mouthwash, toothpaste, or dental floss are cited as examples of formulations from the field of dental care. If the glass ceramics according to the present invention are used in formulations of this type, preservatives and/or other antimicrobially active materials, such as triclosan, may be dispensed with. In addition, the remineralization of the tooth surfaces is accelerated by the donation of Ca and P₂O₅. In addition, through the anti-inflammatory effect of the glass ceramics and/or the glass ceramic powders, gum inflammations are avoided and bleeding gums may be treated effectively. In formulations for dental care and/or oral hygiene, the concentration of the glass ceramic powder is preferably in ranges from 0.1 to 10 weight-percent, especially preferably 1 to 10 weight-percent, particularly preferably 0.1-5 weight-percent, in relation to the total composition.

In formulations for dental care, the glass powder may also be used as an abrasive and for desensitization of the teeth.

The better and rapid formation of the hydroxylapatite coatings in relation to the glass powders known until now and used in formulations for dental care and oral hygiene occurs because an already crystallized material is present. Crystalline subunits may then be deposited on the tooth surface and/or in the tubulin channels. Through the dissolving of the crystals even in the formulations and/or during the application, nanoparticles form, which partially dissolve and/or are also deposited on the tooth surface and/or travel into the tubulin channels. The nanoparticles are especially suitable for being deposited on the tooth surface and building up a mineral coating there. This is particularly the case when the glass ceramic powder contains fluorides. The advantage over glass powders is that these crystalline nanoparticles are fixed in irregularities on the tooth surface and remain there even after washing and therefore long-term hardening of the tooth occurs due to released fluoride and/or calcium and phosphorus.

Particularly because of the formation of hydroxylapatite and nanoparticles, tubular channels may be caused to close. By using the glass ceramic and/or the glass ceramic powder according to the present invention in agents for dental care or oral hygiene, potassium ions or calcium ions may be released, which have an inhibiting effect on tooth pain and may reach tooth nerves or neurons through exposed dentin channels and desensitize them. In this way, pain-free tooth cleaning is made possible and the sensitivity of the teeth to hot-cold changes is reduced (desensitizing of the teeth and/or the tooth nerves/tooth root).

Furthermore, fluoride ions, which harden the tooth, may be released from the glass ceramic and/or the glass ceramic powder. Because of the release of polyphosphates from the glass ceramic and/or the glass ceramic powder, the formation of tartar is reduced and existing tartar is decomposed. A further component which acts against tartar is zinc ions. These may also be a component of the glass ceramic powder and may be released therefrom in aqueous solution.

Another desirable component in toothpastes or tooth creams, gels, and cleaning agents, for example, is a synthetic anionic polymer polycarboxylate (SAPP), which acts as a stabilizer for the polyphosphate, active against tartar, which is released from the glass ceramic and also contributes to blocking the access of painful or pain-causing materials such as sugar to the tooth nerves.

Instead of the polymer polycarboxylates cited, other SAPP types may also be used, preferably at least only partially, such as polysulfonates, polysulfonates, and polyphosphonates, typically up to half of the SAPP content, for example. The different polymers of these types may be produced by reacting an ethylenic unsaturated organic acid such as maleic, crotonic, sorbic, α-chlorsorbic, cinnamic, muconic, itaconic, citraconic, mesaconic, glutaconic, aconitic, angelic, umbellic, or fumaric acid(s) or anhydride(s) with a suitable polymerized ethylenic unsaturated carboxylic, sulfonic, sulfuric, or phosphonic acid, which contains an activated olefinic carbon-carbon double bond and at least one carboxylic, sulfonic, sulfuric acid, or phosphonic acid group. Other olefinic monomers which may be copolymerized with the acids or anhydrides described include vinyl acetate, vinyl chloride, dimethyl maleate, and similar unsaturated monomers, and the copolymers produced will contain a sufficient quantity of acid or neutralized or neutralizable acid groups to make them soluble in water and swellable. Some polycarboxylate copolymers of this type are described in U.S. Pat. Nos. 4,138,477 and 4,183,914.

The fluoride ions released from the glass ceramic in aqueous solution, for example, the oral saliva, contribute to stabilizing the phosphates also released from the glass ceramic against enzymatic attack, while they also provide the compositions with their tooth-hardening and anti-caries properties. In addition to the fluoride ions released from the glass ceramic and/or the glass ceramic powder, the formulation may also have other providers of fluoride ions added to it, for example, water-soluble alkali metal fluorides, such as Na and K fluorides, copper fluorides, such as cuprofluoride, tin fluorides such as stannofluoride, ammonium fluorosilicate, sodium and ammonium fluorozirconate, sodium and potassium fluorophosphate, and aluminum fluorophosphate (mono-, di-, and tri-), and fluorinated sodium-calcium pyrophosphate.

The orally tolerable carrier or base material for the composition according to the present invention, if compositions of this type are toothpastes, which is preferable, normally includes water, humectant, bodying substance, surfactant, or detergent (synthetic surfactant), and a polishing agent. The water used may be any potable water, preferably, however, it is to have a hardness less than 200 ppm calcium carbonate, and especially preferably less than 100 ppm hardness. Demineralized and irradiated water is most preferred. Water and humectant make up the liquid part of the toothpaste. The humectant component of the toothpaste preferably includes a mixture of multiple humectants such as glycerin, sorbite, and polyethylene glycol, which is most preferable, but other mixtures of humectants and individual humectants may also be used. Other humectants which are also usable include propylene glycol and polypropylene glycol. A normal molecular weight range for the polyethylene glycol humectant is 200 to 1000, preferably 400 to 600 or 800, such as 600.

The bodying substance, gelling agent, or thickener of the toothpaste base material may be any agent of this type, but most of these are in the classes of natural and synthetic gums and colloids. Of these, carrageen (Irish moss), xanthan gum, and sodium carboxymethylcellulose may be cited, which are preferable, and also gum tragacanth, starch, polyvinyl pyrrolidone, hydroxyethyl propyl cellulose, hydroxybutyl methyl cellulose, hydroxypropyl methyl cellulose, and hydroxyethyl cellulose (which is available as Natrosol®). Inorganic thickeners, such as colloidal silicic acid, e.g., Syloid®244, and synthetic hectorite, such as Laponite®, which is sold by Laporte Industries, Ltd., may also be used and mixtures of these thickeners are also usable.

The surface active substances or surfactants are normally a water-soluble synthetic surfactant, which is suitable for cleaning the teeth (and the gums) and contributes to bringing the components of the composition which act against tartar and desensitize into contact with the tooth surfaces and penetrating into dentin and pulp, if it is exposed. Synthetic surfactants of this type have valuable foaming properties and also contribute to generating a uniform toothpaste in which the active components are uniformly distributed, so that any quantity of toothpaste corresponding to a fully covered toothbrush (toothbrushful of toothpaste) will contain an active quantity of these materials. The organic, surface-active material is preferably anionic, non-ionic, or ampholytic, and is most preferably anionic. Suitable examples of anionic surfactants are higher alkyl sulfates such as potassium lauryl sulfate, monoglyceride monosulfates of higher fatty acids such as the potassium salt of the mono-sulfated monoglyceride of hydrogenated coconut oil fatty acids. Alkylaryl sulfonates such as potassium dodecylbenzene sulfonate, higher fatty sulfoacetates, esters of higher fatty acids of 1,2-dihydroxypropane sulfonate, and the essentially saturated higher aliphatic acylamides of lower aliphatic amino carboxylic acid compounds, such as those having 12 to 16 carbon atoms in the fatty acid, alkyl or acyl residues and the like. Examples of the latter amides cited are N-lauroyl sarcosine and the potassium salts of N-lauroyl, N-myristoyl, or N-palmitoyl sarcosine, which are to be essentially free of soap or similar higher fatty acid material. Although it is preferable to use potassium surfactants, these are frequently unavailable on the market; in these cases, sodium salts may be used (and sometimes these may even be preferable in the toothpastes described).

Examples of water-soluble non-ionic surfactants are condensation products of ethylene oxide with different hydrogenous compounds, which are reactive therewith and have long hydrophobic chains (e.g., aliphatic chains of approximately 12 to 20 carbon atoms), these condensation products (“ethoxamers”) containing hydrophilic polyoxyethylene components, such as condensation products of poly (ethylene oxide) with fatty acids, fatty alcohols, fatty amides, and other fatty residues, and with propylene oxide and polypropylene oxides (such as Pluronic® materials). Of the synthetic surfactants cited, the sulfates of higher fatty alcohols are preferred (in surfactants of this type and in the other synthetic surfactants cited and overall in this description, the word “higher” identifies, when it is used to identify alkyl groups, fatty acids, etc., those which contain 10 to 20 carbon atoms, preferably 12 to 18, preferably in a linear arrangement).

The glass ceramics and/or glass ceramic powders described here also have, besides the properties described above, the property of functioning as a polishing agent or abrasive in a toothpaste, however, the intention not being that they remove tooth material, but rather only that they remove deposits from the teeth and polish them.

Different other components of toothpaste may be considered as additional active materials or auxiliary materials. These groups include: other compounds active against tartar or calculus such as AHP, PPTA, PBTA, and EHDP, zinc compounds such as zinc chloride, zinc acetate, and zinc oxide, sanquinaria extract; antibacterial substances such as triclosan; buffers for controlling the pH value; bleaches and tooth-whitening agents such as per compounds; protective materials; sweeteners such as potassium (or sodium) saccharin or cyclamate, acesulfame potassium, sucrose, and aspartame; flavorings such as mint (peppermint and spearmint) and menthol; and colors and pigments such as chlorophyll and titanium oxide. Water-soluble active and auxiliary materials of this type in the toothpaste or other oral care agents of the present invention are, if they are in soluble salt form, preferably potassium salts, since it appears that potassium cations increase the desensitization of the tooth nerves through the potassium salts, such as potassium nitrate and potassium citrate, which inhibit tooth pain.

Although it is preferable if the oral care agents of the present invention are toothpastes or gel tooth cleaning agents (including striped tooth cleaning agents), which are brushed on the teeth in order to clean them and prevent tartar formation on them, other forms of oral care agents may also be improved by including the components for preventing tartar formation and desensitizing described in detail here. Since the mechanical forces of the brushing may also be irritating per se and may press irritating chemicals from foods, tartar deposits, candy, and even other tooth care agents into the dentin and the pulp of sensitive teeth, tooth powders, lotions, and liquid tooth cleaning agents may also be significantly improved by including the components for acting against tartar and desensitization cited, although the present invention has its largest application in toothpastes. Other preparations for oral care which are not to be brushed onto the teeth may also contain the components for acting against tartar and desensitization described and these products include mouthwash, antiseptic solutions, chewing gum, tooth treatment agents such as plaque-localizing solutions, and even dental floss and dental tape. In these preparations, which are not toothpaste and are not tooth care agents in gel form, the components of the pyrophosphate acting against tartar are preferably released from the glass ceramic powder or a related compound in a quantity active against tartar and the desensitizing potassium compounds are preferably released from the glass ceramic powder, but also compounds added to the formulation such as potassium nitrate or potassium citrate, will be of a desensitizing quantity. The mouthwash agents normally contain water, alcohol, humectants such as glycerin, sorbite, and/or polyethylene glycol, flavors and sweeteners (not sugar) in addition to the active components cited and the tooth powder contains, besides the glass ceramic powder also acting as a polishing agent among other things, further polishing agents such as zeodent, syloid, santocel, or calcium carbonate. The lotions may contain a carrier or base material made of a rubber or polymer binder such as carrageenan or alginate (preferably potassium alginate) with glass ceramic powder as a filler. Fillers such as calcium carbonate or finely divided silicic acid (in micron size) may also be used. Chewing gums may contain a natural or synthetic elastomer or rubber as a base material. In the other preparations cited, the typical product formulations may also contain the agent active against tartar described and the desensitizing agent according to the present invention or these may be deposited thereon, such as on dental floss and dental tape, and preferably these are potassium-containing glass ceramic powders which release potassium ions.

A special method is preferred for producing toothpaste, since it provides outstanding toothpaste which have the desired pH value and viscosity, and in which the active components have improved stabilities. In this method, the glycerin and polyethylene glycol components of the humectant are first mixed with one another in a typical mixer and then the thickeners, the copolymer, the glass ceramic powder, and possibly additional included alkali metal fluoride and potassium pyrophosphate are dispersed into the humectant mixture by mixing and this mixing is continued until the mixture is a dough or slurry of a smooth appearance, after which the smooth slurry is admixed with the sorbite and water is added and the desensitizing substance(s) are mixed with the diluted slurry. All of these mixing procedures are performed at room temperature in the range from 20 to 30° C. The gel phase produced may then be heated to a temperature in the range from 55 to 75° C., while it is mixed, and the mixing is continued 10 to 30 minutes after the increase temperature in the range given has been reached. When the copolymer is initially in acid form, the basic-acting glass ceramic powder and/or possibly further addition of alkali metal hydroxide, preferably potassium hydroxide, is set to a neutral and/or weakly alkaline pH value in the range from 6 to 8, preferably 7, while mixing, and this mixing is continued for a further 10 to 30 minutes after completion of the addition of the alkali hydroxide. The gel phase obtained is then, when it has been heated, cooled down to a temperature in the range from 35 to 45° C., after which further glass ceramic powder and possibly used polishing agent containing silicic acid is mixed with the gel phase and the mixing is continued for a further 10 to 30 minutes under vacuum in the range from 5 to 100 mm Hg, which leads to the formation of a paste or gel. The last step of the method (except for adding pigments, flavors, sweeteners, or other auxiliary materials) is the mixing of surfactant, preferably anionic surfactant, with the paste or the gel, after which it is mixed for further 3 to 10 minutes under a vacuum of 5 to 50 mm Hg. The product obtained is a consistent, desensitizing toothpaste which acts against tartar and has a viscosity similar to that of normal toothpaste, approximately 100,000 to 500,000 centipoise, a pH in the range from 7 to 11, preferably 7 to 60, 8, for example, is of a pleasing taste (particularly if there is a mint/menthol flavoring), and is not excessively salty. In the above description of the production method, a moisturizing mixing was applied and the addition of flavoring, sweetener, and pigment was not cited. If one or more of the humectants described or any other optimum components are not present in the formulation, the addition steps cited above in which these components are added may be left out. Sweetener and pigment may also be added to the glycerin/polyethylene glycol mixture with the thickener, copolymer, fluoride, and glass ceramic powder and possibly used polyphosphate and the flavoring may be added with the surfactant toward the end of the method.

The toothpastes according to the present invention may be produced using other methods than that described above, but it has been found that the method described provides better toothpastes, because of which it is preferred. In regard to the other embodiments of the present invention, gel tooth cleaning agents may be produced essentially in the same way, using normal adjustments of the formula components and quantities, which are known to those skilled in the art. The production of tooth powder is only a matter of mixing the different active components, and to produce mouthwashes or other liquid preparations, the main active components may be dissolved or dispersed in a suitable liquid medium, typically a water-alcohol medium, and polymers, rubber-like, and insoluble materials are normally left out, although the SAPP may be present. Other types of oral care agents or preparations may be produced according to suitable methods, using suitable additions of the typical active components and the suitable typical supplementary and auxiliary materials during the production method.

An especially preferred use of the glass ceramic powder described is the use in water-free products for oral hygiene and/or dental care such as water-free tooth cream.

The following examples and figures are to explain the present invention and are not to restrict the protective scope. If not otherwise specified, all parts and percentagese-ate to the weight and all temperatures are given in ° C.

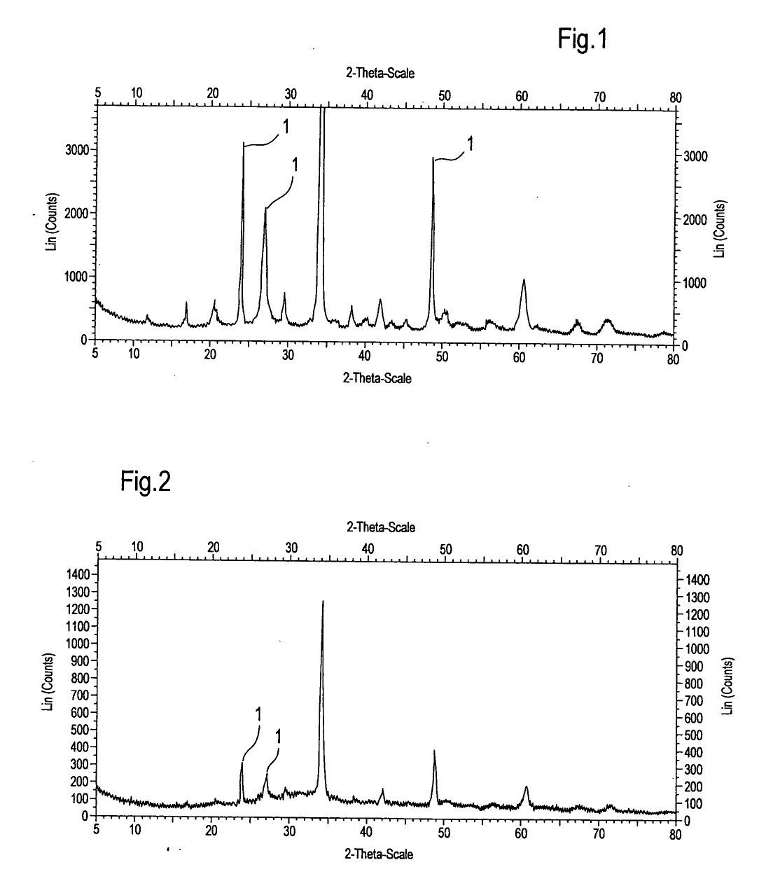

FIG. 1 shows an x-ray diffraction diagram of a starting glass crystallized in powder form having a composition according to exemplary embodiment 1, tempered for 5 hours at 650° C.

FIG. 2 shows an x-ray diffraction diagram of a starting glass crystallized in powder form, tempered for 5 hours at 590° C.

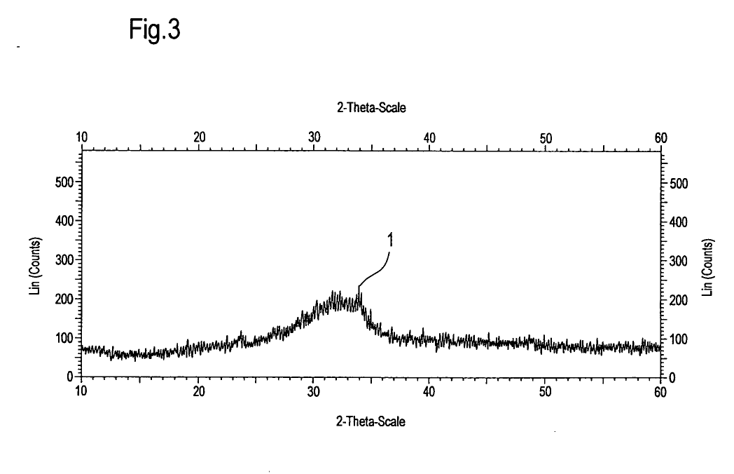

FIG. 3 shows an x-ray diffraction diagram of a starting glass crystallized in powder form, tempered for 5 hours at 560° C.

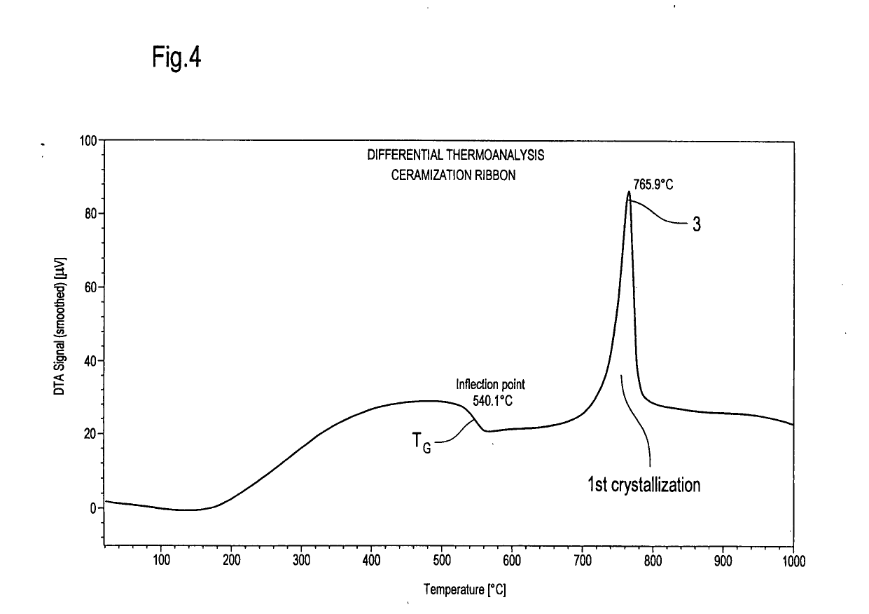

FIG. 4 shows a DTA analysis of a starting glass according to exemplary embodiment 1, ceramicized as a glass block.

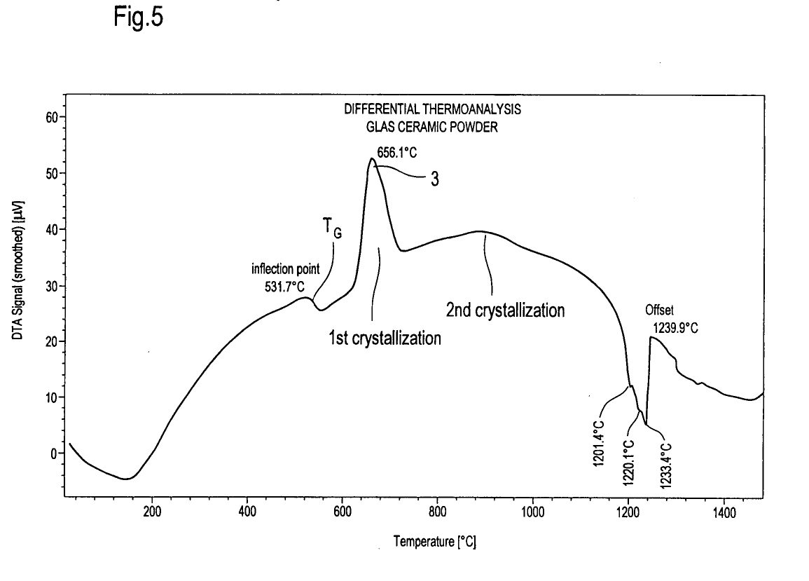

FIG. 5 shows a DTA analysis of a starting glass according to exemplary embodiment 1, ceramicized in powder form.

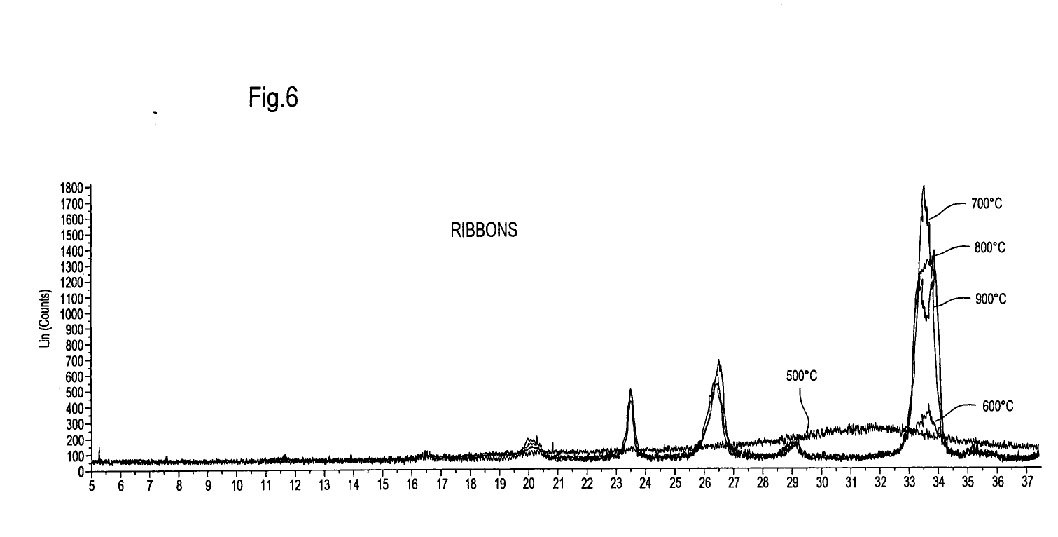

FIG. 6 shows an x-ray diffraction diagram of ceramicized ribbons at different temperatures.

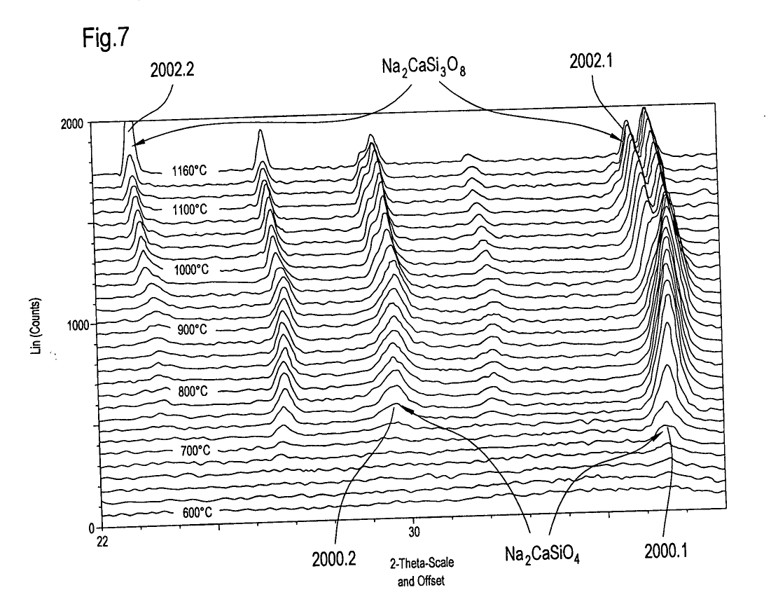

FIG. 7 shows high temperature x-ray diagrams of glass powders having a particle size of 4 μm as a function of the temperature for glass ceramics using a starting glass according to exemplary embodiment 7.

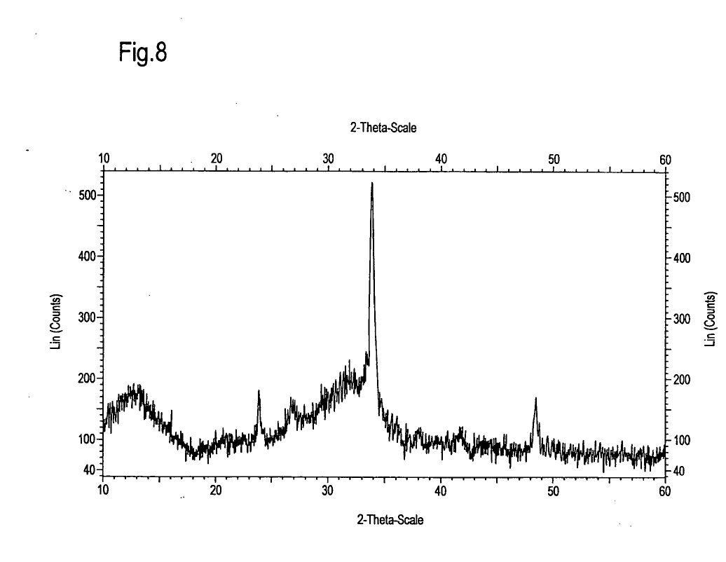

FIG. 8 shows an x-ray diffraction diagram of a crystallized starting glass having a composition according to exemplary embodiment 8, tempered for four hours at 560° C.

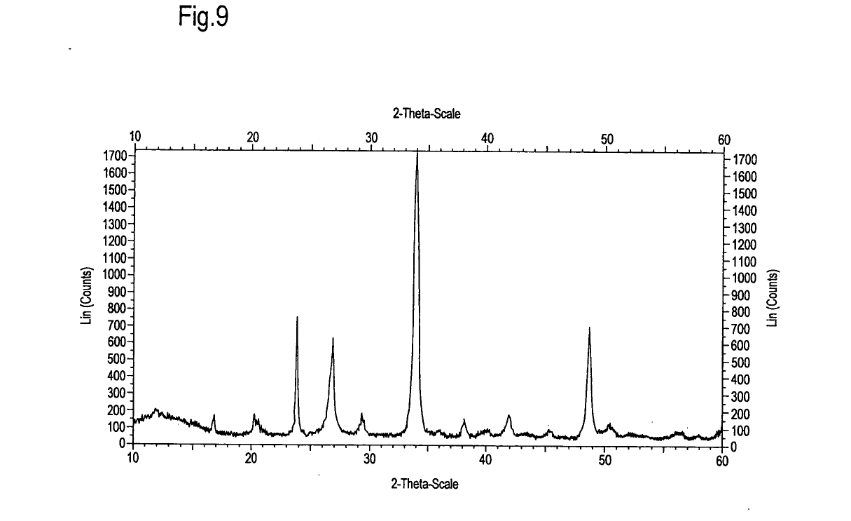

FIG. 9 shows an x-ray diffraction diagram of a crystallized starting glass having a composition according to exemplary embodiment 8, tempered for four hours at 700° C.

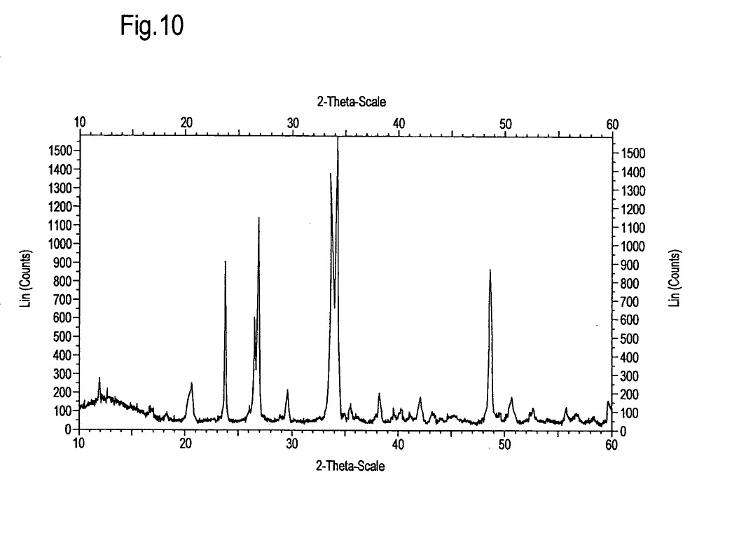

FIG. 10 shows an x-ray diffraction diagram of a crystallized starting glass having a composition according to exemplary embodiment 8, tempered for four hours at 900° C.

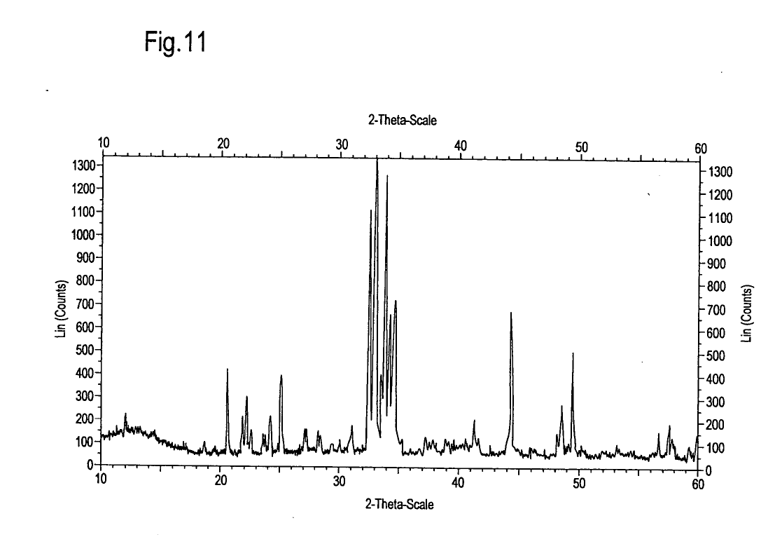

FIG. 11 shows an x-ray diffraction diagram of a crystallized starting glass having a composition according to exemplary embodiment 9, tempered for four hours at 560° C.

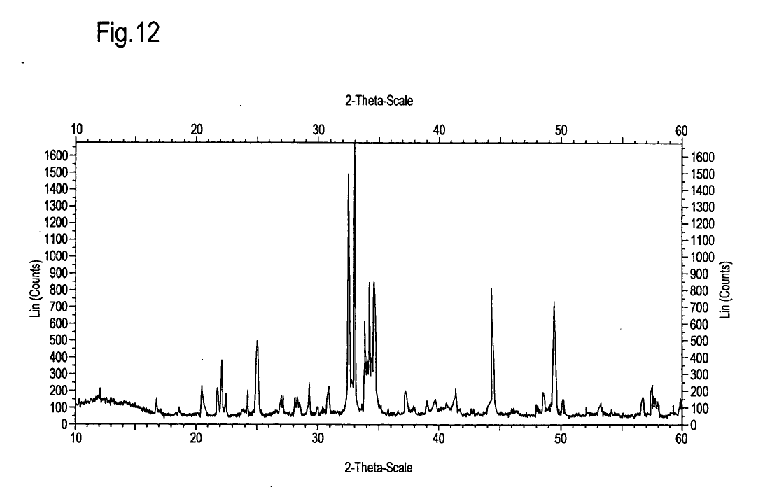

FIG. 12 shows an x-ray diffraction diagram of a crystallized starting glass having a composition according to exemplary embodiment 9, tempered for four hours at 700° C.

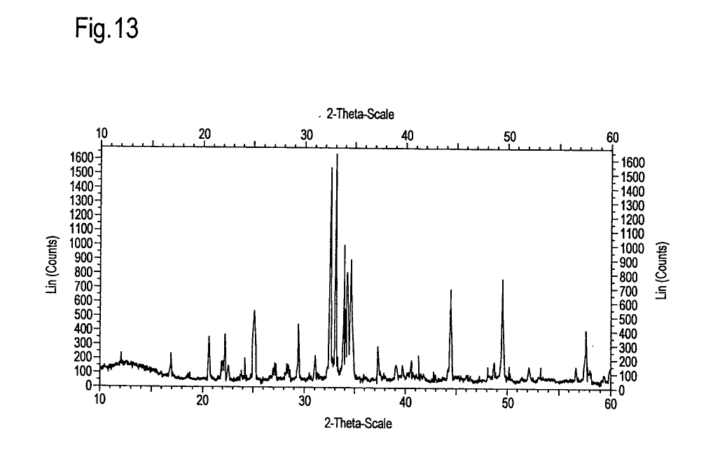

FIG. 13 shows an x-ray diffraction diagram of a crystallized starting glass having a composition according to exemplary embodiment 9, tempered for four hours at 900° C.

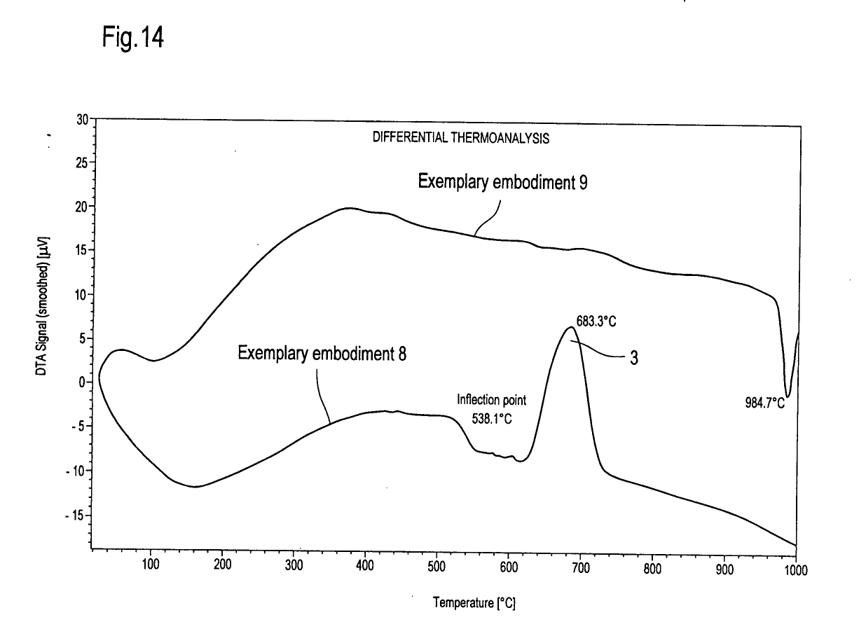

FIG. 14 shows a DTA analysis of a starting glass according to exemplary embodiments 8 and 9, ceramicized as a glass block.

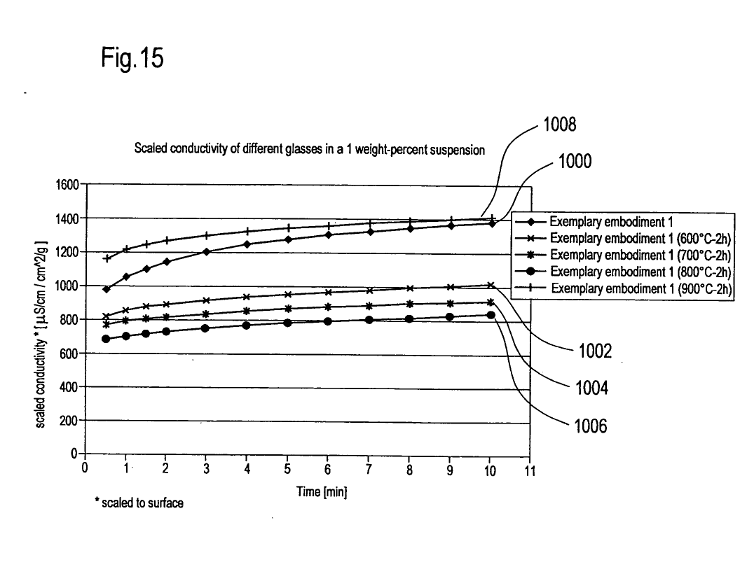

FIG. 15 shows scaled base strengths for a glass ceramic, ceramicized at different temperatures, starting from a starting glass having a composition according to exemplary embodiment 1.

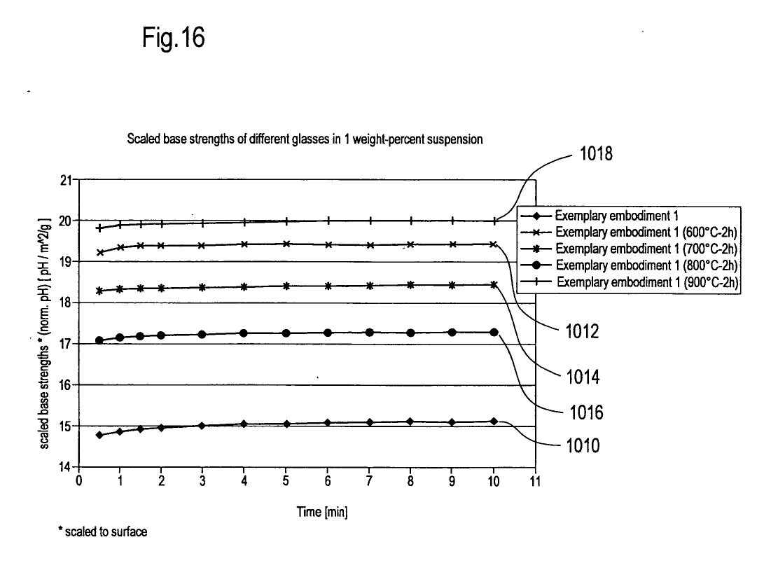

FIG. 16 shows scaled conductivities for glass ceramics, ceramicized at different temperatures, starting from a starting glass having a composition according to exemplary embodiment 1.

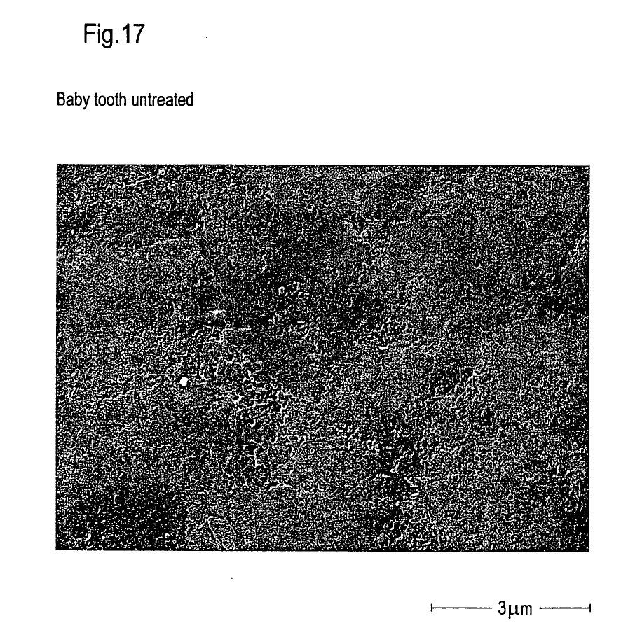

FIG. 17 shows an REM image of an untreated tooth.

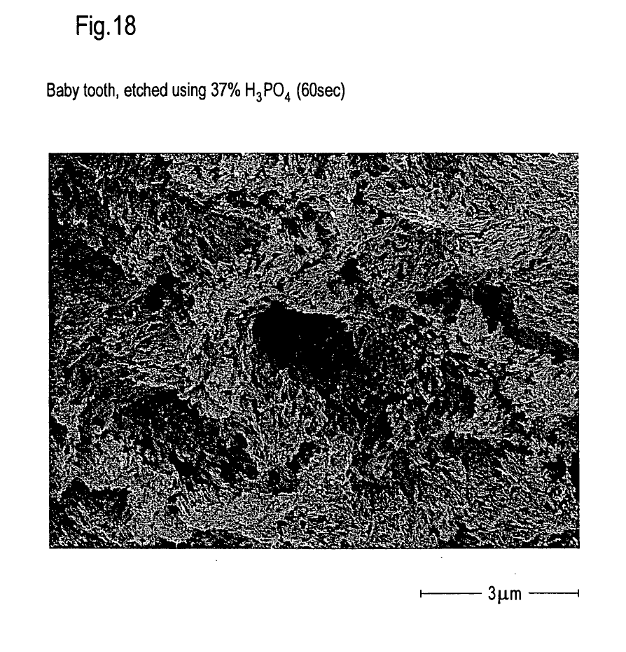

FIG. 18 shows an REM image of a tooth partially dissolved for 60 seconds using 37% phosphoric acid H₃PO₄.

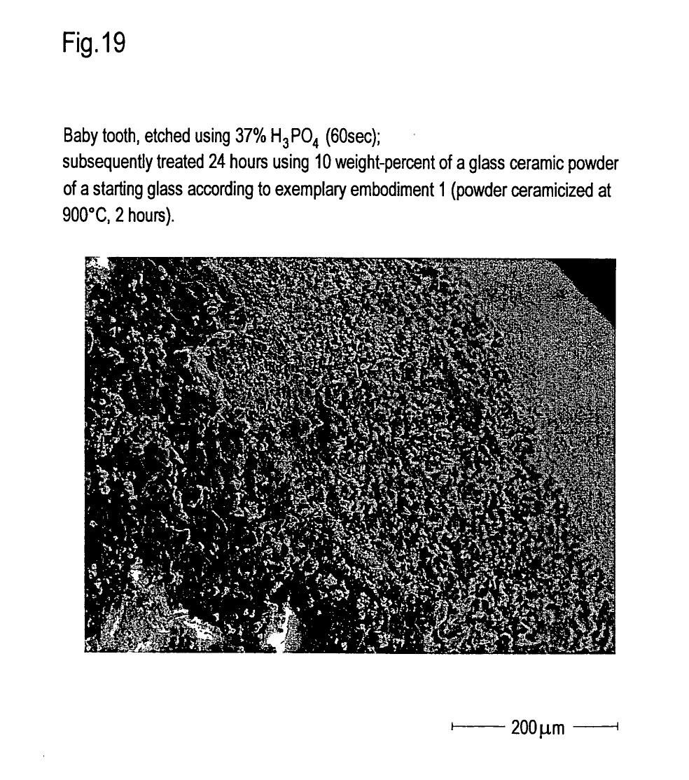

FIG. 19 shows an REM image of the tooth after treatment for 24 hours using a 10% weight-present aqueous suspension of a glass ceramic powder having a starting glass according to exemplary embodiment 1, the powder having been ceramicized for 2 hours at 900° C. The particle size is d₅₀=4 μm. The scale of the image is 200 μm.

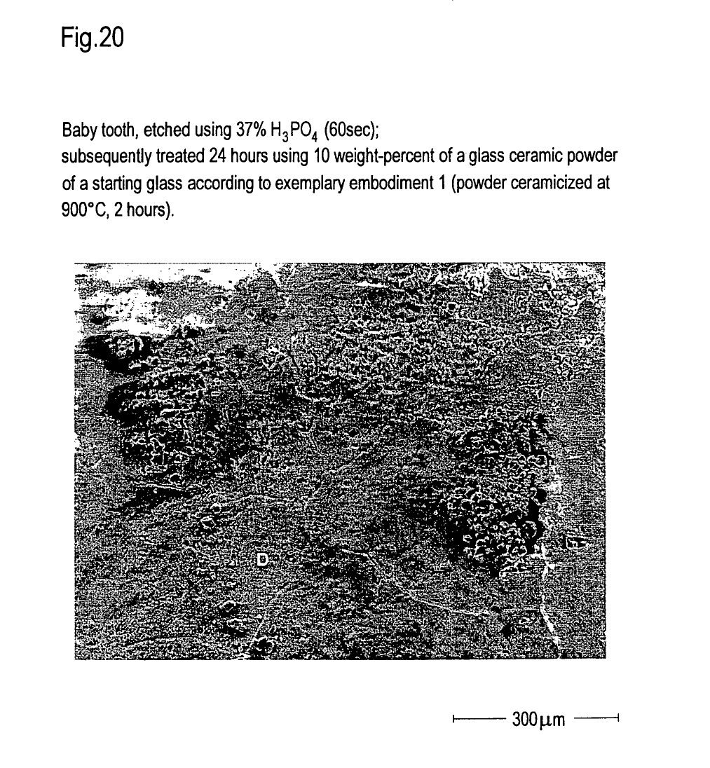

FIG. 20 shows an REM image of a tooth, etched with 37 weight-percent H₃PO₄ for 60 seconds and subsequently treated-for 24 hours with a 10 weight-percent aqueous suspension of a glass ceramic powder, starting from a starting glass according to exemplary embodiment 1, which was obtained through ceramization at 900° C. and tempering for 2 hours. The particle size is d₅₀=4 μm.

FIG. 21 shows an EDX spectrum at point D in FIG. 12.

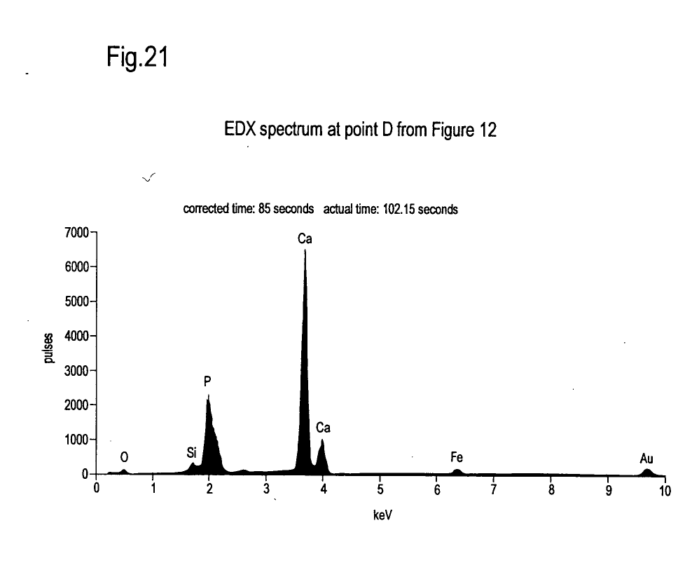

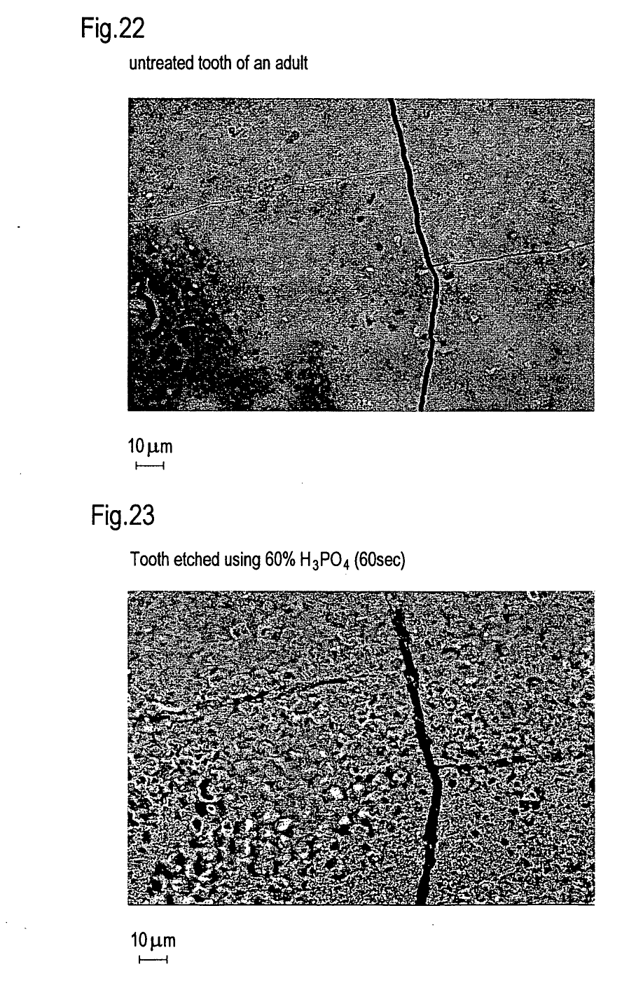

FIG. 22 shows an untreated tooth of an adult.

FIG. 23 shows a tooth etched using 60% H₃PO₄ (60 seconds).

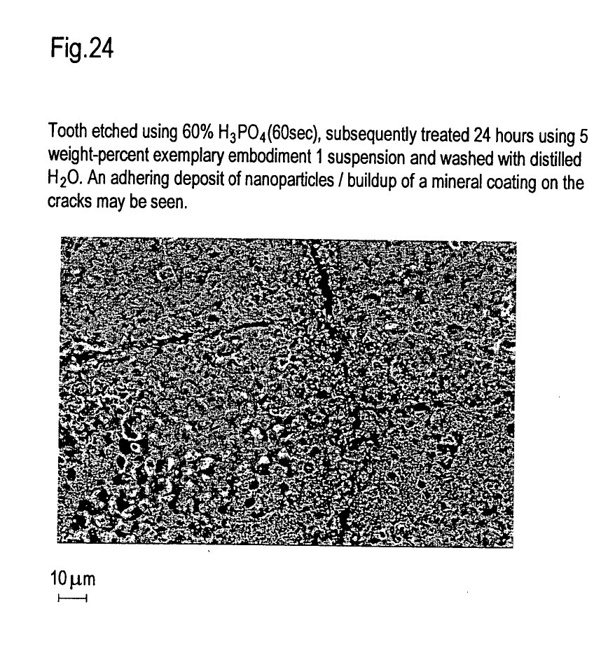

FIG. 24 shows a tooth etched using 60% H₃PO₄ (60 seconds), subsequently treated for 24 hours using 5% suspension of exemplary embodiment 1.

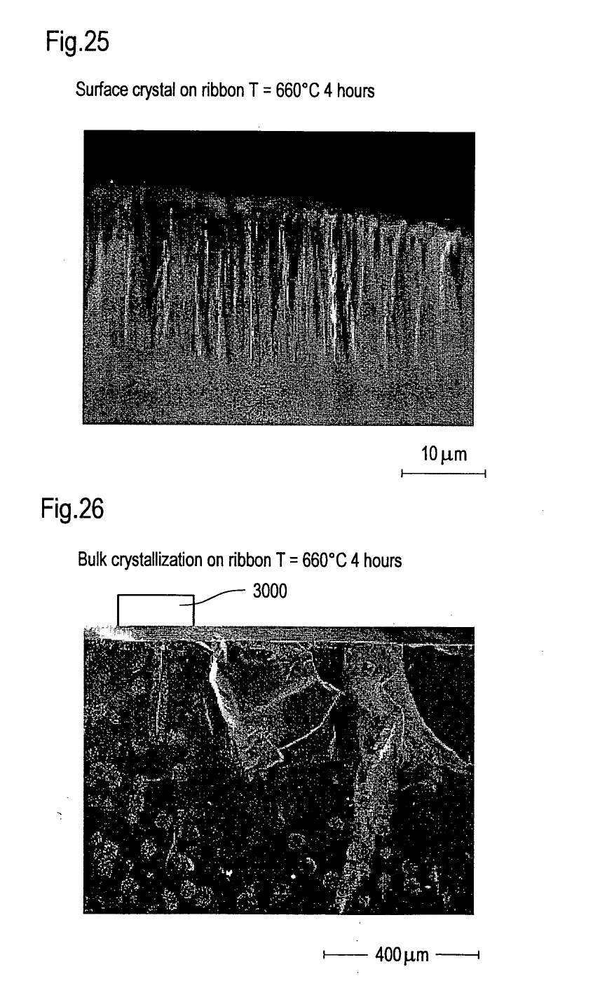

FIG. 25 shows an REM image of the surface crystals on the surface of a glass ceramic which was obtained by tempering a starting glass according to exemplary embodiment 1 at 660° C. for 4 hours.

FIG. 26 shows an REM image of a section through a glass ceramic which was obtained through bulk crystallization by tempering at T=660° C. for 4 hours.

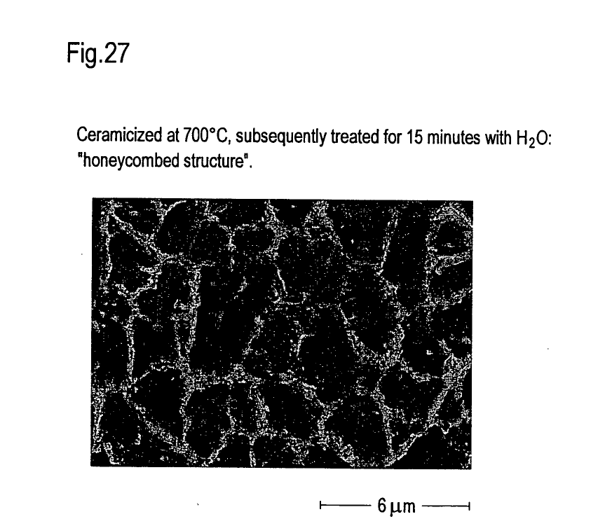

FIG. 27 shows the surface of a glass ceramic ribbon ceramicized at 700° C. and subsequently treated with water for 15 minutes.

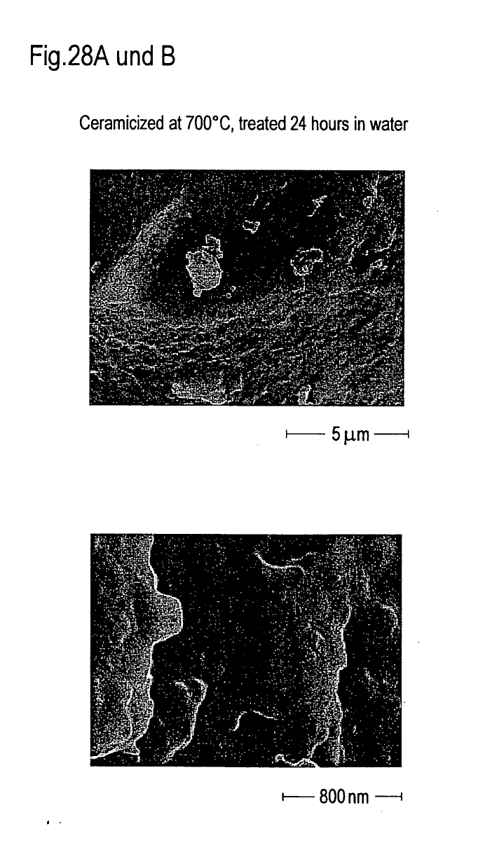

FIG. 28A-B shows the surface of a glass ceramic powder, ceramicized at 700° C. and subsequently treated for 24 hours in water.

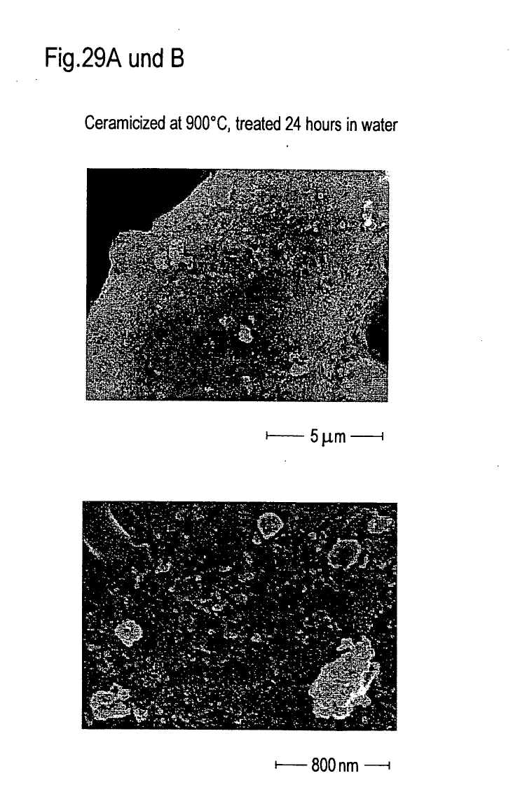

FIG. 29A-B shows the surface of a glass ceramic powder, ceramicized at 900° C., and subsequently treated for 24 hours in water.

First, the production of the glass ceramic and/or the glass ceramic powder is to be described starting from the starting glass.

A glass was melted from the raw materials. The melt was performed in platinum crucibles at 1550° C. The melt was subsequently shaped into ribbons. These ribbons were processed further, using dry milling, into powder having a particle size d₅₀=4 μm.

The compositions of the starting glasses in weight-percent are specified in Table 1.

TABLE 1
Compositions (synthetic values) [weight-percent]

| | Example | | | | | | | | | | |
|---|---|---|---|---|---|---|---|---|---|---|---|
| | 1 | 2 | 3 | 4 | 5 | 6 | 7 | 8 | 9 | 10 | 11 |
| SiO₂ | 46.0 | 35 | 46 | 50 | 40 | 59 | 45 | 44.9 | 35 | 45 | 65 |
| Al₂O₃ | 0 | 0 | 0 | 0 | 0 | 0 | 1 | 0 | 0 | 0 | 0 |
| CaO | 25.0 | 29 | 20 | 10 | 25 | 20 | 25 | 24.5 | 29.0 | 23.5 | 10.0 |
| MgO | 0 | 0 | 5 | 0 | 0 | 0 | 0 | 0 | 0 | 0 | 0 |
| Na₂O | 25.0 | 30 | 20 | 25 | 25 | 20 | 24 | 24.5 | 29.5 | 24.5 | 20.0 |
| K₂O | 0 | 0 | 5 | 0 | 0 | 0 | 0 | 0 | 0 | 0 | 0 |
| P₂O₅ | 4.0 | 6 | 0 | 15 | 0 | 1 | 7 | 0 | 0 | 0 | 5.0 |
| Ag₂O | 0 | 0 | 0 | 0 | 0 | 0 | 0 | 0.1 | 0.1 | 0 | 0 |
| ZnO | 0 | 0 | 0 | 0 | 0 | 0 | 0 | 0 | 0 | 1.0 | 0 |

Examples 2 and 9 tend strongly toward crystallization even during the melting of the glass. It is therefore necessary to cool down these starting glasses especially rapidly. If a complete ceramization occurs even during the melting of the glass, the glass ceramic may be subjected to another tempering at the temperature specified in order to obtain the crystal phases described here.

FIGS. 1-3 show the x-ray diffraction diagrams of starting glasses according to exemplary embodiment 1 in Table 1 crystallized in powder form, tempered for 5 hours at 650° C. (FIG. 1), 590° C. (FIG. 2), and 560° C. (FIG. 3). The reduction in intensity of the orders of diffraction 1, which originate from the crystal phases, may be seen clearly, which is equivalent to a sinking crystal component in the glass ceramic. The intensity peaks 1 may be assigned to Na₂CaSiO₄/Na₂OCaOSiO₂ and Na₂Ca₂Si₃O₈ crystal phases, for example.

At higher temperatures, a recrystallization occurs, as may be seen in FIG. 6. At temperatures >900° C., Ca silicates may also form.

FIGS. 4 and 5 show the DTA thermoanalysis of the starting glass according to exemplary embodiment 1 in Table 1 ceramicized as ribbon (FIG. 4) and the starting glass ceramicized in powder form (FIG. 5) using heating rates of 10 K/minute. The crystallization peak 3 for the crystal phase, which is shifted to lower temperatures for the starting glass ceramicized in powder, may be seen clearly.

The exothermic reaction of the recrystallization may also be seen weakly in FIG. 5.

High-temperature x-ray diagrams for a glass ceramic powder which was obtained from a starting glass according to exemplary embodiment 7 are shown in FIG. 7 as a function of the temperature. The x-ray measurements were recorded during the heating. At higher temperatures greater than 900° C., recrystallization occurs. At these temperatures, Ca silicates may also form. In FIG. 7, the Na₂CaSiO₄ phase assignable according to the JCPDS databank is identified with 2000.1 and 2000.2, and the Na₂Ca₂Si₃O₈ phase assignable according to the JCPDS databank is identified with 2002.1 and 2002.2. As may be seen in FIG. 7, the Na₂Ca₂Si₃O₈ phase is first formed at temperatures above approximately 900° C.

The properties of the glass ceramics produced in different ways, starting from the starting glass according to example 1 in Table 1, are reproduced in Table 2.

TABLE 2
properties of glass ceramics according
to exemplary embodiment 1

| | Tempering | | Crystalline | JCPDS |
|---|---|---|---|---|
| | time | Crystallite size | primary phases | databank |
| Powder | 5 hours | <0.5 | Na₂Ca₂Si₃O₈/ | 12-0671/ |
| 580° C. | | | Na₂CaSiO₄ | 24-10696 |
| | | | Na₂Ca₂(SiO₃)₃ | |
| Powder | 5 hours | <1 | Na₂Ca₂Si₃O₈/ | 12-0671/ |
| 650° C. | | | Na₂CaSiO₄ | 24-10696 |
| | | | Na₂Ca₂(SiO₃)₃ | |
| Powder | 5 hours | <1 | Na₂Ca₂Si₃O₈/ | 12-0671/ |
| 700° C. | | | Na₂CaSiO₄ | 24-10696 |
| | | | Na₂Ca₂(SiO₃)₃ | |
| Ribbons | 5 hours | >100 μm | Na₂Ca₂Si₃O₈/ | 12-0671/ |
| 700° C. | | | Na₂CaSiO₄ | 24-10696 |
| | | | Na₂Ca₂(SiO₃)₃ | |
| Ribbons | 2 hours | >20 μm in | Na₂Ca₂Si₃O₈/ | 12-0671/ |
| 600° C. | | volume | Na₂CaSiO₄ | 24-10696 |
| | | | Na₂Ca₂(SiO₃)₃ | |

Table 3 shows the antibacterial effect of a glass ceramic powder, which was tempered for 5 hours at 580° C., having a grain size of 4 μm.

TABLE 3
Antibacterial effect of the powder according to European Pharmacopeia
(3rd edition): exemplary embodiment 1 (grain size 4 μm)

| | | E. | P. | S. | C. | A. |
|---|---|---|---|---|---|---|
| | | coli | aeruginosa | aureus | albicans | niger |
| Start | | 290,000 | 270,000 | 250,000 | 300,000 | 250,000 |
| 2 | days | 900 | 1800 | 800 | <100 | 2000 |
| 7 | days | <100 | 200 | <100 | 0 | 2000 |
| 14 | days | 0 | 0 | 0 | 0 | 0 |
| 21 | days | 0 | 0 | 0 | 0 | 0 |
| 28 | days | 0 | 0 | 0 | 0 | 0 |

In the skin tolerability test, i.e., occlusive tests over 24 hours, the skin irritations were established.

In Table 4, crystalline primary phases of Na—Ca silicates systems are specified in detail in tabular form, the general formula
x Na₂O.y CaO.z SiO₂

having been used as a basis and the numbers being specified for x, y, and z.

TABLE 4
Crystalline primary phases of Na≡Ca silicates systems

| Na₂O (x) | CaO (y) | SiO₂ (z) |
|---|---|---|
| 1 | 3 | 6 |
| 1 | 1 | 5 |
| 1 | 2 | 3 |
| 1 | — | 2 |
| 3 | — | 8 |
| 2 | 3 | 6 |
| 2 | — | 2 |
| 0 | 1 | 1 |
| 1 | 0 | 1 |

In the following, the results will be shown for glass ceramics which were obtained from starting glasses according to exemplary embodiments 8 and 9.

FIGS. 8-10 show the x-ray diffraction diagrams of starting glasses according to exemplary embodiment 8 in Table 1 crystallized in powder form, tempered for 4 hours at 560° C. (FIG. 8), 700° C. (FIG. 9), and 900° C. (FIG. 10). The phase which may be determined from the intensity peak is a Na—Ca silicate, specifically Na₆Ca₃Si₆O₁₈ (JCPDS 77-2189) as the crystalline phase. The change of the Na—Ca ratio with increasing temperature may be seen clearly.

FIGS. 11-13 show the x-ray diffraction diagrams of starting glasses according to exemplary embodiment 9 in Table 1 crystallized in powder form, tempered for 4 hours at 560° C. (FIG. 11), 700° C. (FIG. 12), and 900° C. (FIG. 13).

In FIGS. 11-13, two Na—Ca silicates Na₂CaSiO₄ (JCPDS 73-1726) and Na₂Ca₂SiO₇ (JCPDS 10-0016), as well as silicon phosphate SiP₂O₇ (JCPDS 39-0189) and cristobalite SiO₂ (JCPDS 82-0512) may be identified as the crystalline primary phases. In the samples produced at 700° C. and 900° C. which are shown in FIGS. 12 and 13, a further crystalline phase is contained, specifically silver phosphate AgPO₃ (JCPDS 11-0641). The component of this phase is greater in the sample produced at 900° C. than in the sample produced at 700° C.

FIG. 14 shows the DTA thermoanalysis of starting glass according to exemplary embodiments 8 and 9 in Table 1 ceramicized as ribbon using heating rates of 10 K/minute. The crystallization phase 3 for the crystal phase may be seen clearly for the exemplary embodiment 8. The glass ceramic which originates from the starting glass according to exemplary embodiment 9 is a glass ceramic crystallized already from the melt. In this case, a strongly exothermic signal may no longer be observed in the DTA, since the further crystallization and/or recrystallization only releases a little further heat. This may be attributed to the fact that the starting glass in this exemplary embodiment already tends to spontaneous crystallization during melting.

Table 5 shows the antibacterial effect of a glass ceramic powder which was tempered at 560° C., starting from a starting glass according to exemplary embodiment 8, having a grain size of 4 μm.

TABLE 5
Antibacterial effect of the powder according to European Pharmacopeia
(3rd edition): exemplary embodiment 8 (grain size 4 μm)

| | | E. | P. | S. | C. | A. |
|---|---|---|---|---|---|---|
| | | coli | aeruginosa | aureus | albicans | niger |
| Start | | 290,000 | 270,000 | 250,000 | 300,000 | 250,000 |
| 2 | days | 700 | 2000 | 500 | <100 | 2000 |
| 7 | days | 0 | 0 | 0 | 0 | 0 |
| 14 | days | 0 | 0 | 0 | 0 | 0 |
| 21 | days | 0 | 0 | 0 | 0 | 0 |
| 28 | days | 0 | 0 | 0 | 0 | 0 |

Table 6 shows the antibacterial effect of a glass ceramic powder which was tempered at 900° C., starting from a starting glass according to exemplary embodiment 9, having a grain size of 4 μm.

TABLE 6
Antibacterial effect of the powder according to European
Pharmacopeia (3rd edition): exemplary embodiment 9
(grain size 4 μm)

| | | E. | P. | S. | C. | A. |
|---|---|---|---|---|---|---|
| | | coli | aeruginosa | aureus | albicans | niger |
| Start | | 290,000 | 270,000 | 250,000 | 300,000 | 250,000 |
| 2 | days | 0 | 0 | 0 | 0 | 0 |
| 7 | days | 0 | 0 | 0 | 0 | 0 |
| 14 | days | 0 | 0 | 0 | 0 | 0 |
| 21 | days | 0 | 0 | 0 | 0 | 0 |
| 28 | days | 0 | 0 | 0 | 0 | 0 |

In Table 7, crystalline primary phases found in the samples produced are specified in detail in tabular form, the general formula
x Na₂O.y CaO.z SiO₂
having been used as a basis and the numbers being specified for x, y, and z.

Besides the Na—Ca phases, a silicon phosphate phase was found. In addition, at high temperatures above 700° C., a silver phosphate phase was found.

TABLE 7
crystalline primary phases of the glass ceramics exemplary
embodiments 8 and 9

| Na₂O (x) | CaO (y) | SiO₂ (z) | Ag₂O | P₂O₅ | Note |
|---|---|---|---|---|---|
| | | | 1 | 1 | from 700 |
| 3 | 3 | 6 | | | |
| 1 | 1 | 1 | | | |
| 1 | 2 | 1 | | | |
| | | 1 | | 1 | |
| 2 | 1 | 3 | | | |

In Table 8, the pH values and the conductivities of a 1% suspension of a glass ceramic powder which is made of a base glass according to exemplary embodiment 7 in Table 1 are specified for different tempering conditions-for the production of the glass ceramic. The tempering times and the tempering temperatures are specified in the tempering conditions. Depending on the tempering time and tempering temperature, different primary crystalline phases result in the glass ceramic.

TABLE 8
pH value and conductivity of a glass ceramic powder

| | | | | | After 24 | |
|---|---|---|---|---|---|---|
| | After 15 | | After 60 | | hours | |
| Tempering | minutes | Conductivity | minutes | Conductivity | pH | Conductivity |
| conditions | pH value | (μS/cm) | pH value | (μS/cm) | value | (μS/cm) |
| Untreated | 11.3 | 695 | 11.3 | 900 | 11.7 | 1672 |
| 500° C. - 2 hours | 11.1 | 526 | 11.2 | 623 | 11.4 | 1054 |
| 600° C. - 2 hours | 11.2 | 571 | 11.2 | 686 | 11.5 | 1130 |
| 700° C. - 2 hours | 11.2 | 576 | 11.2 | 679 | 11.5 | 1007 |
| 800° C. - 2 hours | 11.2 | 619 | 11.3 | 746 | 11.5 | 1138 |
| 900° C. - 2 hours | 11.3 | 859 | 11.4 | 949 | 11.5 | 1288 |

In FIGS. 15 and 16, the pH values, i.e., the base strengths, and the conductivities of a 1% suspension of a glass ceramic powder which is made of a base glass according to exemplary embodiment 1 in Table 1 are specified for different tempering conditions in the production of the glass ceramic. The ceramization conditions are specified for the particular glass powder in brackets. The curves of these variables are shown over time. The scaling is in relation to the specific surface area of the particle (m²/g). The tempering times and the tempering temperatures are specified in the tempering conditions. Depending on the tempering time and tempering temperature, different primary crystalline phases result in the glass ceramic.

In FIG. 15, the curves of the scaled conductivity for a glass ceramic powder which is made of a base glass according to exemplary embodiment 1 in Table 1 are shown over time. In this case, the curve 1000 describes the conductivity of an unceramicized starting glass, 1002 describes the conductivity of a starting glass tempered at 600° C. for 2 hours, 1004 describes the conductivity of a starting glass tempered at 700° C. for 2 hours, 1006 describes the conductivity of a starting glass tempered at 800° C. for 2 hours, and 1008 describes the conductivity of a starting glass tempered at 900° C. for 2 hours.

It may be seen from FIG. 15 that the conductivity, i.e., the component of mobile ions in the solution, may be adjusted outstandingly via the ceramization.

In FIG. 16, the curves of the scaled base strengths for a glass ceramic powder which is made of a base glass according to exemplary embodiment 1 in Table 1 are shown over time. In this case, the curve 1010 describes the base strength of an unceramicized starting glass, 1012 describes the base strength of a starting glass tempered at 600° C. for 2 hours, 1014 describes the base strength of a starting glass tempered at 700° C. for 2 hours, 1016 describes the base strength of a starting glass tempered at 800° C. for 2 hours, and 1018 describes the base strength of a starting glass tempered at 900° C. for 2 hours.

It may be seen from FIG. 16 that the basicity, i.e., the implementation of the quantity of total exchanged ions, may be adjusted outstandingly via the ceramization. The pH value, which is scaled to the specific surface area, represents a measure of the total ions exchanged in this case.

It may be seen from FIG. 16 that in the glass ceramic which was ceramicized for 2 hours at 900° C., for example, a significantly higher reactivity may be achieved than in the unceramicized glass.

From the observation of FIGS. 15 and 16, i.e., the scaled pH and scaled conductivity, it may be recognized that in the scaled basicity of the glass ceramic having the identification numbers 1008, 1018 (900° C./2 hours), a significantly increased formation of minerals, such as hydroxylapatite, among other things, occurs in comparison to the unceramicized glass. The scaled conductivities, i.e., the mobile ions in the solution, are equal in a first approximation, but the number of ions exchanged is multiple times higher in the glass ceramic. In this case, reference is made to the logarithmic representation of the y-axis. Since the ions exchanged are no longer mobile in the solution and therefore may no longer contribute to the conductivity, they have precipitated out of the solution.

Quantification of the precipitated crystals using analytical methods is only possible with difficulty because of the partially formed nanocrystals.

In Table 9, the ionic permeability of unceramicized powder and glass ceramic powder which are made of a glass according to exemplary embodiment 7 is shown in Table 1 as the starting glass in 1% suspension. The glass ceramic powder was produced by tempering at 650° C. for 4 hours.

TABLE 9
ionic permeability (1% suspension, units mg/l)

| | | Powder | Powder |
|---|---|---|---|
| | | ceramicized | ceramicized |
| | | 650° C./4 | 900° C./4 |
| | Unceramicized | hours | hours |
| Na | 96.7 mg/l | 63.2 mg/l | 90.7 mg/l |
| Ca | 29.9 mg/l | 21.5 mg/l | 26.8 mg/l |
| Si | 63.5 mg/l | 40.3 mg/l | 59.2 mg/l |
| P | 0.22 mg/l | 0.67 mg/l | 0.19 mg/l |

REM images of a baby tooth are shown in FIGS. 17 through 20, in FIG. 17, an untreated tooth which was partially dissolved using 37% phosphoric acid. The partially dissolved tooth is shown in FIG. 18. FIGS. 19 and 20 show the tooth after a treatment using a 10 weight-percent aqueous suspension of a glass ceramic powder in tris buffer. The glass ceramic powder originates from a starting glass according to exemplary embodiment 1. This starting glass was tempered at 900° C. for 2 hours. The particle size is d₅₀=4 μm. The tris buffer used is: tris-hydroxyl-methyl-amino methane. The structure of the mineral coating may be recognized both in FIG. 19 and in FIG. 20. A mineral coating of this type is, for example, a hydroxylapatite coating. As may be inferred from the EDX spectrum recorded at position D in FIG. 21, a Ca—Si—P coating, i.e., a hydroxylapatite phase is implemented at this point. The buildup of a mineral coating has a remineralizing effect for the tooth.

In FIGS. 22 through 24, REM images of a tooth of an adult are shown, in FIG. 22 an untreated tooth which was partially dissolved using 60% phosphoric acid. The tooth, which was partially dissolved for 60 seconds, is shown in FIG. 23. FIG. 24 shows the tooth after a treatment using a 5 weight-percent aqueous suspension of a glass ceramic powder. The glass ceramic powder originates from a starting glass according to exemplary embodiment 1. This starting glass was tempered at 900° C. for 2 hours. The particle size is d₅₀=4 μm. The buildup of the mineral coating and the adhering deposits of nanoparticles may be recognized in FIG. 24. A mineral coating of this type is, for example, a hydroxylapatite coating. The buildup of a mineral coating has a remineralizing effect for the tooth and is implemented on the cracks, as shown in FIG. 24.

In the following, raster electron microscope images (REM images) of glass ceramics which were obtained through crystallization of a starting glass according to exemplary embodiment 1 will be shown.

FIG. 25 shows an REM image of the surface of a glass ceramic which was obtained through crystallization from a starting glass according to exemplary embodiment 1 at a temperature of T=660° C. and tempering for 4 hours. The surface crystals on the ribbon may be seen clearly. Parts of the surface crystals may be water-soluble, so that these are dissolved out upon treatment with water and a honeycombed structure remains. Furthermore, specific phases may be dissolved out of this crystalline surface as nanoparticles which are important for, among other things, oral care applications, i.e., uses of the glass ceramics of the present invention in the fields of dental and oral care. Furthermore, the crystalline surface shown in this figure has light-scattering properties, which may be exploited for specific applications.

While the surface structure of the glass ceramic was shown in FIG. 25, an REM image of the crystallization inside the glass block, i.e., the bulk crystallization, is shown in FIG. 26. FIG. 25 is a detail of FIG. 26. The detail is identified in FIG. 26 with 3000. The glass ceramic shown in FIGS. 25 and 26 was obtained through tempering at T=660° C. for 4 hours. The crystallites formed may be recognized in FIG. 26 as round spots. The crystals formed in the bulk have light-scattering properties which may be exploited for specific applications. In FIGS. 25 and 26, the crystallization was performed in the glass block (ribbon).

FIG. 25 and FIG. 26 show a cross-section through the surface of the block and/or ribbon. FIG. 25 is a detail of FIG. 26 and shows the surface in detail.

In FIG. 27, the surface of a glass ceramic ribbon which was obtained through ceramization of a starting glass according to exemplary embodiment 1 by tempering at 700° C. for 4 hours is shown. Subsequently, the glass ceramic was treated for 15 minutes using H₂O. The easily soluble crystalline phases, essentially including Na—Ca silicate, were dissolved out. A “honeycombed” structure remains, as may be seen clearly in FIG. 27.

The surface of a glass ceramic powder which was obtained from a starting glass according to exemplary embodiment 1 through ceramization at 700° C. for 4 hours in powder is shown in FIGS. 28A and B. The surface shown was obtained by treating the glass ceramic powder for 24 hours using water. Furthermore, surface irregularities may be seen in FIGS. 28A and 28B. As may be inferred from the figures, the surface is relatively homogeneous and hardly shows the formation of nanoparticles.

The surface a glass ceramic powder which was obtained from a starting glass according to exemplary embodiment 1 through ceramization at 900° C. for 4 hours in powder is shown in FIGS. 29A and 29B. In contrast to the smooth surface obtained at lower temperatures, as is shown in FIGS. 28A and 28B, the nanocrystals dissolved out and a porous structure of the surface may be recognized in FIGS. 29A and 29B.

The crystalline nanoparticles are poorly water-soluble. The nanoparticles were formed in the tempering step and have been dissolved out of the surface.

The dissolved-out nanoparticles are important for, among other things, oral care applications, since they have a desensitizing effect on tooth nerves. The desensitizing effect is achieved in that the nanoparticles may seal the tubular channels.

In Table 10, the crystallite sizes of glass ceramics starting from a starting glass according to exemplary embodiment 1 are specified for different temperatures. The crystallite sizes are the average crystallite size of a crystallite size distribution determined from the half width of the x-ray reflection of the intensity peak 1 of the x-ray diffraction diagram, as shown in FIGS. 1 through 3, for example.

TABLE 10
Half widths of the glass ceramics, determined according
to the Scherrer equation using a starting glass according
to exemplary embodiment 1 for different tempering temperatures
at a tempering time of 2 hours.

| | | | |
|---|---|---|---|
| Tempering | 600° C. | 700° C. | 900° C. |
| temperature | | | |
| Crystallite size | 43 nm | 64 nm | 101 nm |

In the following, a formulation of a tooth cream, which has 31.06 weight-percent of a glass ceramic powder according to the present invention of the exemplary embodiment 1 shown in Table 1, having 46.0 weight-percent SiO₂, 25.0 weight-percent CaO, 5.0 weight-percent Na₂O, and 4 weight-percent P₂O₅, will be specified:

TABLE 10
Composition of a tooth cream having glass ceramic powder
according to the present invention

| Component | Weight-percent |
|---|---|
| Glycerin (99.3% pure) | 10.00 |
| Polyethylene glycol 600 | 3.00 |
| Carrageenan (90% active) | 0.85 |
| Sodium saccharin | 0.40 |
| Sodium fluoride | 0.24 |
| Titanium dioxide, FDA quality | 0.5 |
| SAPP** | 11.5 |
| (copolymer of maleic acid anhydride with vinyl methyl | |
| ether, average MW approximately 70,000 according to | |
| vapor pressure osmometry) | |
| Water*** | 17.8 |
| Sorbite solution (70% aqueous solution) | 22.5 |
| Sodium lauryl sulfate | 1.2 |
| Peppermint flavoring**** | 0.95 |
| Glass ceramic powder using a starting glass of a base | 31.06 |
| composition according to exemplary embodiment 1 in | |
| Table 1, ceramicized at 580° C. for 4 hours, grain size | |
| and/or particle size d₅₀ = 4 μm | |

*Viscarin TP-206, produced by Marine Colloids Division of FMC Corporation
**Gantrez S-97, liquid, produced by CAF Corporation
***demineralized and irradiated (treatment using UV rays)
****includes menthol

The glass ceramic powder according to the present invention is also especially suitable for use in water-free formulations, such as water-free tooth creams, etc.

The present invention provides for the first time a glass ceramic powder and a glass ceramic which may be used in the fields of dental care and oral hygiene in an advantageous way. The glass ceramic according to the present invention and/or the glass ceramic powder according to the present invention are especially suitable as an additive in formulations for oral hygiene and/or dental care, in tooth creams, for example. By introducing glass ceramic powder instead of glass powders, in formulations for dental care, for example, the mechanical properties are greatly improved. Tribological activation and therefore higher reactivity of the powder occurs due to milling. Glass ceramic powders having particle sizes <10 μm, preferably <5 μm, especially preferably <2 μm, particularly preferably <1 μm, are especially suitable for dental applications. In contrast to glass powders which release ions in formulations for dental care primarily only via ion exchange, the crystalline phases of a glass ceramic powder partially dissolve in aqueous systems. In addition, secondary nanoparticles are generated and the reactivity is greatly increased in comparison to glass powders.

1. A method of using glass ceramic in oral hygiene and/or dental care, comprising:
applying a composition to the mouth of a user, said composition comprising

| | |
|---|---|
| SiO₂ | 30 to [[−]] 70 weight-percent; |
| Na₂O | 0 to [[−]] 40 weight-percent; |
| K₂O | 0 to [[−]] 40 weight-percent; |
| CaO | 5 to [[−]] 40 weight-percent; |
| MgO | 0 to [[−]] 40 weight-percent; |
| Al₂O₃ | 0 to [[−]] 5 weight-percent; |
| P₂O₅ | 0 to [[−]] 20 weight-percent; |
| B₂O₃ | 0 to [[−]] 5 weight-percent; |
| ZnO | 0 to [[−]] 10 weight-percent; and |

0 to 30 weight-percent XFy, wherein X is selected from the group consisting of Na, K, Mg, and Ca, and y is equal to 1 or 2, and the sum of Na₂O+K₂O+CaO is greater than 10 weight-percent and less than 70 weight-percent.

2. A use of a method of using glass ceramic in oral hygiene and/or dental care comprising:
applying a composition to the mouth of a user, said composition comprising

| | |
|---|---|
| SiO₂ | 30 to [[−]] 70 weight-percent; |
| Na₂O | 0 to [[−]] 40 weight-percent; |
| K₂O | 0 to [[−]] 40 weight-percent; |
| CaO | 5 to [[−]] 40 weight-percent; |
| MgO | 0 to [[−]] 40 weight-percent; |
| Al₂O₃ | 0 to [[−]] 5 weight-percent; |
| P₂O₅ | 1 to [[−]] 20 weight-percent; |
| B₂O₃ | 0 to [[−]] 5 weight-percent; |
| ZnO | 0 to [[−]] 10 weight-percent; and |

0 to 30 weight-percent XFy, wherein X is selected from the group consisting of Na, K, Mg, and Ca, and wherein y is equal to 1 or 2.

3. A method of using glass ceramic in oral hygiene and/or dental care, comprising:
applying a composition to the mouth of a user, said composition comprising:

| | |
|---|---|
| SiO₂ | 30 to [[−]] 60 weight-percent; |
| K₂O | 0 to [[−]] 30 weight-percent; |
| Na₂O | 5 to [[−]] 30 weight-percent; |
| CaO | 5 to [[−]] 30 weight-percent; |
| P₂O₅ | 2 to [[−]] 10 weight-percent; |
| ZnO | 0 to [[−]] 10 weight-percent; and |

0 to 30 weight-percent XFy, wherein X is selected from the group consisting of Na, K, Mg, or and Ca, and wherein y is equal to 1 or 2.

4. A method of using glass ceramic in oral hygiene and/or dental care, comprising:
applying a composition to the mouth of a user, said composition comprising:

| | | |
|---|---|---|
| SiO₂ | 30-60 | weight-percent; |
| K₂O | 5-30 | weight-percent; |
| Na₂O | 0-30 | weight-percent; |
| CaO | 5-30 | weight-percent; |
| P₂O₅ | 2-10 | weight-percent; |
| ZnO | 0-10 | weight-percent; and |

0 to 30 weight-percent XFy, wherein X is selected from the group consisting of Na, K, Mg, and Ca, and wherein y is equal to 1 or 2.

5. The method as in claim 1, wherein the sum of Na and K is 5 to 40 weight-percent.

6. The method as in claim 1, wherein said composition is effective for a use selected from the group consisting of tooth remineralization, treatment of bleeding gums, reducing mouth odor, treating periodontitis, treating caries, desensitizing teeth, treating inflammations, and preventing tartar formation.

7. The method as in claim 2, wherein said composition is effective for a use selected from the group consisting of tooth remineralization, treatment of bleeding gums, reducing mouth odor, treating periodontitis, treating caries, desensitizing teeth, treating inflammations, and preventing tartar formation.

8. The method as in claim 3, wherein said composition is effective for a use selected from the group consisting of tooth remineralization, treatment of bleeding gums, reducing mouth odor, treating periodontitis, treating caries, desensitizing teeth, treating inflammations, and preventing tartar formation.

9. The method as in claim 4, wherein said composition is effective for a use selected from the group consisting of tooth remineralization, treatment of bleeding gums, reducing mouth odor, treating periodontitis, treating caries, desensitizing teeth, treating inflammations, and preventing tartar formation.

10-13. (canceled)

14. The method as in claim 1, wherein the crystalline primary phase of the glass ceramic includes alkali-alkaline earth silicates and alkaline earth silicates.

15. The method as in claim 14, wherein the crystalline primary phase includes sodium-calcium silicates and calcium silicates.

16. The method as in claim 1, wherein said composition further comprises metal ions selected from the group consisting of Ag, Au, I, Cu, and Ce, said metal ions comprising less than 2 weight-percent.

17. The method as in claim 1, further comprising producing the glass ceramic from a starting glass through ceramization in powder form before applying said composition to the mouth of a user.

18. The method as in claim 1, further comprising producing the glass ceramic through ceramization of bulk glass and/or ribbon with subsequent milling before applying said composition to the mouth of a user.

19. The method as in claim 18, wherein the glass ceramic has a particle size that is larger than 100 μm.

20. A formulation for dental care or oral hygiene, comprising:
at least 0.1 to 30 weight-percent of a glass ceramic, said glass ceramic having:

| | |
|---|---|
| SiO₂ | 30 to [[−]] 70 weight-percent; |
| Na₂O | 0 to [[−]] 40 weight-percent; |
| K₂O | 0 to [[−]] 40 weight-percent; |
| CaO | 5 to [[−]] 40 weight-percent; |
| MgO | 0 to [[−]] 40 weight-percent; |
| Al₂O₃ | 0 to [[−]] 5 weight-percent; |
| P₂O₅ | 1 to [[−]] 20 weight-percent; |
| B₂O₃ | 0 to [[−]] 5 weight-percent; |
| ZnO | 0 to [[−]] 10 weight-percent; and |

0 to 30 weight-percent XFy, wherein X is selected from the group consisting of Na, K, Mg, and Ca, and wherein y is equal to 1 or 2.

21. A formulation for dental care or oral hygiene, comprising:
at least 0.1 to 30 weight-percent of a glass ceramic having the following compositions:

| | |
|---|---|
| SiO₂ | 30 to [[−]] 60 weight-percent; |
| K₂O | 0 to [[−]] 30 weight-percent; |
| Na₂O | 5 to [[−]] 30 weight-percent; |
| CaO | 5 to [[−]] 30 weight-percent; |
| P₂O₅ | 2 to [[−]] 10 weight-percent; |
| ZnO | 0 to [[−]] 10 weight-percent; and |

0 to 30 weight-percent XFy, wherein X is selected from the group consisting of Na, K, Mg, and Ca, and wherein y is equal to 1 or 2.

22. A formulation for dental care or oral hygiene, comprising:
at least 0.1 to 30 weight-percent of a glass ceramic having the following composition:

| | |
|---|---|
| SiO₂ | 30 to [[−]] 60 weight-percent; |
| K₂O | 5 to [[−]] 30 weight-percent; |
| Na₂O | 0 to [[−]] 30 weight-percent; |
| CaO | 5 to [[−]] 30 weight-percent; |
| P₂O₅ | 2 to [[−]] 10 weight-percent; |
| ZnO | 0 to [[−]] 10 weight-percent; and |

0 to 30 weight-percent XFy, wherein X is selected from the group consisting of Na, K, Mg, and Ca, and wherein y is equal to 1 or 2.

23. The formulation as in claim 20, wherein the formulation is usable in dental care for tooth remineralization, caries prophylaxis, and desensitizing teeth.

24. The formulation as in claim 21, wherein the formulation is usable in dental care for tooth remineralization, caries prophylaxis, and desensitizing teeth.

25. The formulation as in claim 22, wherein the formulation is usable in dental care for tooth remineralization, caries prophylaxis, and desensitizing teeth.

26. The formulation as in claim 20, wherein the formulation is usable in toothpaste, mouthwash, dental floss, and care agents for third teeth.

27. The formulation as in claim 21, wherein the formulation is usable in toothpaste, mouthwash, dental floss, and care agents for third teeth.

28. The formulation as in claim 22, wherein the formulation is usable in toothpaste, mouthwash, dental floss, and care agents for third teeth.
